(19) United States
(12) Patent Application Publication
(10) Pub. No.: US 2011/0237659 A1
(43) Pub. Date: Sep. 29, 2011

(54) CHROMENONE DERIVATIVES AS TRPV3 ANTAGONISTS

(75) Inventors: Sachin Sundarlal Chaudhari, Navi Mumbai (IN); Abraham Thomas, Navi Mumbai (IN); Ashok Bhausaheb Kadam, Navi Mumbai (IN); Sachin Vasantrao Dhone, Usmanabad (IN); Bharat Gangadhar Adik, Ahmednagar (IN); Neelima Khairatkar-joshi, Thane (w) (IN); Vidya Ganapati Kattige, Thane (W) (IN)

(73) Assignee: GLENMARK PHARMACEUTICALS S.A., La Chaux-de-Fonds (CH)

(21) Appl. No.: 13/126,003

(22) PCT Filed: Nov. 6, 2009
(86) PCT No.: PCT/IB09/07353

§ 371 Date: Apr. 26, 2011

Related U.S. Application Data
(60) Provisional application No. 61/138,456, filed on Dec. 17, 2008.
(60) Provisional application No. 61/171,265, filed on Apr. 21, 2009.

(30) Foreign Application Priority Data
Nov. 17, 2008 (IN) 2420/MUM/2008
Mar. 23, 2009 (IN) 664/MUM/2009

(51) Int. Cl. A61K 31/353; C07D 311/36; A61P 29/00; A61P 25/00; A61P 27/02; A61P 17/00; A61P 9/00; A61P 13/10; A61P 13/00; A61P 1/04; A61P 11/00; A61P 11/06; A61P 21/02; A61P 1/08; A61P 25/14; A61P 25/24; A61P 19/02; A61P 3/10; A61P 3/04; A61P 17/14; A61P 27/16; A61P 35/00; A61P 25/22

(52) U.S. Cl. 514/456; 549/403

(57) ABSTRACT

The present invention provides transient receptor potential vanilloid (TRPV) modulators of formula (I). In particular, compounds described herein are useful for treating or preventing diseases, conditions and/or disorders modulated by TRPV3. Also provided herein are processes for preparing compounds described herein, intermediates used in their synthesis, pharmaceutical compositions thereof, and methods for treating or preventing diseases, conditions and/or disorders modulated by TRPV3.

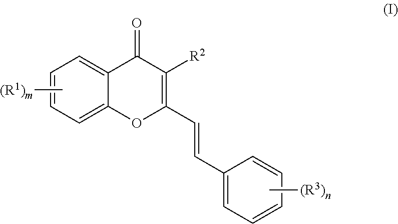

### RELATED APPLICATIONS

This application claims the benefit of Indian Provisional Applications 2420/MUM/2008, filed on Nov. 17, 2008; 664/MUM/2009, filed on Mar. 23, 2009; and U.S. Provisional Applications 61/138,456, filed on Dec. 17, 2008; 61/171,265, filed on Apr. 21, 2009; all of which are hereby incorporated by reference in their entirety.

### TECHNICAL FIELD

The present patent application relates to chromenone derivatives as TRPV3 antagonists.

### BACKGROUND

Movement of ions across cellular membranes is carried out by specialized proteins. TRP channels are one large family of non-selective cation channels that function to help regulate ion flux and membrane potential. TRP channels are subdivided into 6 sub-families including the TRPV family. TRPV3 is a member of the TRPV class of TRP channels.

TRPV3 is a calcium permeable nonselective cation channel. In addition to calcium ions, TRPV3 channels are permeable to other cations, for example sodium. Thus, TRPV3 channels modulate membrane potential by modulating the flux of cations such as calcium and sodium ions. TRPV3 receptors are mechanistically distinct from voltage-gated calcium channels. Generally, voltage-gated calcium channels respond to membrane depolarization and open to permit an influx of calcium from the extracellular medium that result in an increase in intracellular calcium levels or concentrations. In contrast, TRP channels which are non-selective, long lasting, produce more prolonged changes in ion concentration and are ligand gated (modulated by chemicals such as 2-aminoethoxydiphenyl borate [2-APB], vanilloids and heat). These mechanistic differences are accompanied by structural differences among voltage-gated and TRP channels. Thus, although many diverse channels act to regulate ion flux and membrane potential in various cell types and in response to numerous stimuli, it is important to recognize the significant structural, functional, and mechanistic differences among different classes of ion channels.

TRPV3 proteins are thermosensitive channels expressed in skin cells (Peier et al. Science (2002), 296, 2046-2049) and dorsal root ganglion, trigeminal ganglion, spinal cord and brain (Xu et al. Nature (2002), 418, 181-185; Smith et al. Nature (2002), 418, 186-188). In a keratinocyte cell line, stimulation of TRPV3 leads to release of inflammatory mediators including Interleukin-1 Thus TRPV3 may also play an important role in regulating inflammation and pain that results from the release of inflammatory stimuli. Particular TRPV3 proteins that may be used in screening assays, as described herein, to identify compounds that modulate a function of TRPV3 include, but are not limited to human TRPV3, mouse TRPV3, rat TRPV3 and Drosophila TRPV3. US2004/0009537 (the '537 application) disclosed sequences corresponding to human, mouse, and Drosophila TRPV3. For example, SEQ ID Nos 106 and 107 of the '537 application correspond to the human nucleic acid and amino acid sequences, respectively. SEQ ID Nos 108 and 109 of the '537 application correspond to the mouse nucleic acid and amino acid sequences, respectively.

TRPV3 function has been basically implicated in the reception and transduction of pain. Accordingly, it would be desirable to identify and make compounds that can modulate one or more functions of TRPV3.

WO 2007/056124, WO 2008/140750 and WO 2008/033564 disclose TRPV3 modulators, in particular antagonists, for treatment of various diseases mediated TRPV3.

In efforts to discover better analgesics, there still exists a need for therapeutic treatment of diseases, conditions and/or disorders modulated by TRPV3.

### SUMMARY OF THE INVENTION

The present patent application relates to compounds of the general formula (I)

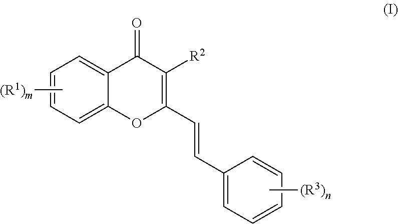

wherein,

at each occurrence, R¹ is independently selected from hydrogen, nitro, cyano, halogen, —OR^{a}, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heterocyclic group, —NR⁴R⁵, —S(O)ₚNR⁴R⁵, and —S(O)ₚR⁴;

R² is selected from hydrogen, halogen, substituted or unsubstituted aryl, substituted or unsubstituted heteroaryl, or substituted or unsubstituted heterocyclic group; wherein substituent(s) are independently selected from halogen, nitro, cyano, —NR⁴R⁵, substituted or unsubstituted alkyl, substituted or unsubstituted alkenyl, substituted or unsubstituted alkoxy, substituted or unsubstituted haloalkyl, substituted or unsubstituted haloalkyloxy, substituted or unsubstituted cycloalkyl, substituted or unsubstituted aryl, substituted or unsubstituted heterocyclic group, and substituted or unsubstituted heteroaryl;

at each occurrence, R³ may be same or different and is selected from nitro, cyano, halogen, —OR^{a}, substituted or unsubstituted alkyl, substituted or unsubstituted alkenyl, substituted or unsubstituted haloalkyl, substituted or unsubstituted cyanoalkyl, substituted or unsubstituted cyanoalkyloxy, substituted or unsubstituted cycloalkyl, substituted or unsubstituted aryl, substituted or unsubstituted heterocyclic group, and substituted or unsubstituted heteroaryl;

at each occurrence, R^{a} is independently selected from hydrogen, substituted or unsubstituted alkyl, linear or branched chain alkyl, substituted or unsubstituted haloalkyl, substituted or unsubstituted cyanoalkyl, substituted or unsubstituted alkenyl, substituted or unsubstituted cycloalkyl, substituted or unsubstituted alkoxyalkyl, substituted or unsubstituted aryl, substituted or unsubstituted heteroaryl, substituted or unsubstituted heterocyclic group, substituted or unsubstituted cycloalkylalkyl, substituted or unsubstituted arylalkyl, substituted or unsubstituted heteroarylalkyl, and substituted or unsubstituted heterocyclylalkyl;

at each occurrence, R⁴ and R⁵ are independently selected from hydrogen, substituted or unsubstituted alkyl, substituted or unsubstituted alkenyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic group, and substituted or unsubstituted heterocyclylalkyl;

‘n’ is an integer selected from 0 to 5, both inclusive;

‘m’ is an integer selected from 0 to 4, both inclusive; and

at each occurrence, ‘p’ is an integer selected from 0 to 2, both inclusive.

It should be understood that the formula (I) structurally encompasses all geometrical isomer, stereoisomer, enantiomer and diastereomer and pharmaceutically acceptable salt that may be contemplated from the chemical structure of the genus described herein.

According to one preferred embodiment, the compound has the formula:

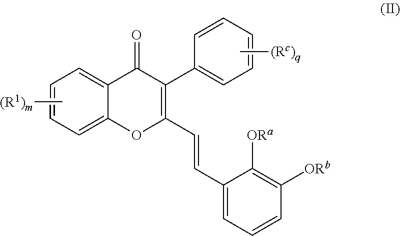

wherein,

at each occurrence, R¹ is independently selected from hydrogen, nitro, cyano, halogen, —OR^{a}, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heterocyclic group, —NR⁴R⁵, —S(O)ₚNR⁴R⁵, and —S(O)ₚR⁴;

R^{a} is selected from hydrogen, substituted or unsubstituted alkyl, linear or branched chain alkyl, substituted or unsubstituted haloalkyl, substituted or unsubstituted cyanoalkyl, substituted or unsubstituted alkenyl, substituted or unsubstituted cycloalkyl, substituted or unsubstituted alkoxyalkyl, substituted or unsubstituted aryl, substituted or unsubstituted heteroaryl, substituted or unsubstituted heterocyclic group, substituted or unsubstituted cycloalkylalkyl, substituted or unsubstituted arylalkyl, substituted or unsubstituted heteroarylalkyl, and substituted or unsubstituted heterocyclylalkyl;

R^{b} is selected from hydrogen, substituted or unsubstituted alkyl, substituted or unsubstituted alkenyl, substituted or unsubstituted haloalkyl, substituted or unsubstituted cycloalkyl, substituted or unsubstituted aryl, substituted or unsubstituted heterocyclic group, and substituted or unsubstituted heteroaryl;

at each occurrence, R^{c} is independently selected from hydrogen, nitro, cyano, halogen, —OR^{a}, substituted or unsubstituted alkyl, substituted or unsubstituted haloalkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heterocyclic group, —NR⁴R⁵, —S(O)ₚNR⁴R⁵, and —S(O)ₚR⁴;

at each occurrence, R⁴ and R⁵ are independently selected from hydrogen, substituted or unsubstituted alkyl, substituted or unsubstituted alkenyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic group, and substituted or unsubstituted heterocyclylalkyl;

‘m’ is an integer selected from 0 to 4, both inclusive;

at each occurrence, ‘p’ is an integer selected from 0 to 2, both inclusive; and

‘q’ is an integer selected from 0 to 5, both inclusive.

It should be understood that the formula (II) structurally encompasses all geometrical isomer, stereoisomer, enantiomer and diastereomer and pharmaceutically acceptable salt that may be contemplated from the chemical structure of the genus described herein.

According to one embodiment, specifically provided are compounds of the formula (II), in which R¹ is hydrogen or halogen (example F, Cl or Br); and ‘m’ is 1 or 2.

According to another embodiment, specifically provided are compounds of the formula (II), in which R^{a} is hydrogen, linear or branched chain alkyl (example methyl, iso-butyl, iso-pentyl, or neo-pentyl), substituted or unsubstituted haloalkyl (example 3,3,3-trifluoropropyl), substituted or unsubstituted cycloalkyl (example cyclopentyl) or substituted or unsubstituted cycloalkylalkyl (example cyclopropylmethyl or cyclobutylmethyl).

According to another embodiment, specifically provided are compounds of the formula (II), in which R^{b} is hydrogen, linear or branched chain alkyl (example methyl, iso-butyl, iso-pentyl or neo-pentyl), substituted or unsubstituted haloalkyl (example difluoromethyl), substituted or unsubstituted cycloalkyl or substituted or unsubstituted cycloalkylalkyl.

According to another embodiment, specifically provided are compounds of the formula (II), in which R^{c} is cyano, haloalkyl (example trifluoromethyl) or haloalkoxy (example trifluoromethoxy); and ‘q’ is 0 or 1.

Below are the representative compounds, which are illustrative in nature only and are not intended to limit to the scope of the invention.
2-[(E)-2-(2-Cyclopropylmethoxy-3-methoxyphenyl)-1-ethenyl]-3-(4-trifluoromethoxy-phenyl)-4H-4-chromenone,
2-[(E)-2-(2-Cyclopropylmethoxy-3-methoxyphenyl)-1-ethenyl]-3-(4-trifluoromethyl-phenyl)-4H-4-chromenone,
4-{2-[(E)-2-(2-Cyclopropylmethoxy-3-methoxyphenyl)-1-ethenyl]-4-oxo-4H-3-chromenyl}benzonitrile,
4-(2-{(E)-2-[2-(Cyclopropylomethoxy)-3-(difluoromethoxy)phenyl]vinyl}-4-oxo-4H-3-chromenyl}benzonitrile,
4-{2-[(E)-2-(2-Cyclopentyloxy-3-methoxyphenyl)-1-ethenyl]-4-oxo-4H-3-chromenyl}benzonitrile,
4-{2-[(E)-2-(2-Hydroxy-3-methoxyphenyl)-1-ethenyl]-4-oxo-4H-3-chromenyl}-benzonitrile,
4-{2-[(E)-2-[(E)-2-(2,2-Dimethylpropoxy)-3-methoxyphenyl]-1-ethenyl]-4-oxo-4H-3-chromenyl}benzonitrile,
4-{2-[(E)-2-(2-Isobutoxy-3-methoxyphenyl)-1-ethenyl]-4-oxo-4H-3-chromenyl}benzonitrile,
4-(2-{(E)-2-[3-Methoxy-2-(3,3,3-trifluoropropoxy)phenyl]-1-ethenyl}-4-oxo-4H-3-chromenyl}benzonitrile,
4-{2-[(E)-2-(2-Cyclopropylmethoxy-3-methoxyphenyl)-1-ethenyl]-7-fluoro-4-oxo-4H-3-chromenyl}benzonitrile,
4-{2-[(E)-2-(2-Cyclopentyloxy-3-methoxyphenyl)-1-ethenyl]-7-fluoro-4-oxo-4H-3-chromenyl}benzonitrile,
4-{7-Fluoro-2-[(E)-2-(3-methoxy-2-neopentyloxyphenyl)-1-ethenyl]-4-oxo-4H-3-chromenyl}benzonitrile,
4-{7-Fluoro-2-[(E)-2-(2-isobutoxy-3-methoxyphenyl)-1-ethenyl]-4-oxo-4H-3-chromenyl}benzonitrile,
4-(7-Fluoro-2-{(E)-2-[3-methoxy-2-(3,3,3-trifluoropropoxy)phenyl]-1-ethenyl}-4-oxo-4H-3-chromenyl)benzonitrile,
4-{2-[(E)-2-(2-Cyclopropylmethoxy-3-methoxyphenyl)-1-ethenyl]-6-fluoro-4-oxo-4H-3-chromenyl}benzonitrile,
4-{2-[(E)-2-(2-Cyclopentyloxy-3-methoxyphenyl)-1-ethenyl]-6-fluoro-4-oxo-4H-3-chromenyl}benzonitrile,
4-{2-[(E)-2-(2-Cyclobutylmethoxy-3-methoxyphenyl)-1-ethenyl]-6-fluoro-4-oxo-4H-3-chromenyl}benzonitrile,
4-{6-Fluoro-2-[(E)-2-(2-isopentyloxy-3-methoxyphenyl)-1-ethenyl]-4-oxo-4H-3-chromenyl}benzonitrile,
4-{6-Fluoro-2-[(E)-2-(2-isobutoxy-3-methoxyphenyl)-1-ethenyl]-4-oxo-4H-3-chromenyl}benzonitrile
4-{6-Fluoro-2-[(E)-2-(2,2-dimethylpropoxy)-3-methoxyphenyl]-1-ethenyl]-4-oxo-4H-3-chromenyl}benzonitrile,
4-{6-Chloro-2-[(E)-2-(2-cyclopropylmethoxy-3-methoxyphenyl)-1-ethenyl]-4-oxo-4H-3-chromenyl}benzonitrile,
4-{2-[(E)-2-(2-Cyclopropylmethoxy-3-methoxyphenyl)-1-ethenyl]-6,8-difluoro-4-oxo-4H-3-chromenyl}benzonitrile, and
4-{2-[(E)-2-[2-Cyclopentyloxy-3-methoxyphenyl)-1-ethenyl]-6,7-difluoro-4-oxo-4H-3-chromenyl}benzonitrile or

an analog, tautomer, regiomer, geometrical isomer, stereoisomer, enantiomer, diastereomer or pharmaceutically acceptable salt thereof.

The present patent application also provides a pharmaceutical composition that includes at least one compound of described herein and at least one pharmaceutically acceptable excipient (such as a pharmaceutically acceptable carrier or diluent). Preferably, the pharmaceutical composition comprises a therapeutically effective amount of at least one compound described herein. The compound(s) present in the composition may be associated with a pharmaceutically acceptable excipient (such as a carrier or a diluent) or may be diluted by a carrier, or enclosed within a carrier which may be in the form of a capsule, sachet, paper or other container.

The compounds and pharmaceutical compositions described herein are useful in the treatment of diseases, conditions and/or disorders modulated by TRPV3 receptors.

The present patent application further provides a method of treating a disease, condition and/or disorder modulated by TRPV3 receptors in a subject in need thereof by administering to the subject one or more compounds described herein in the amount effective to cause inhibition of such receptor.

Also provided herein are processes for preparing compounds described herein.

### DETAILED DESCRIPTION

The present patent application provides chromenone derivatives, which may be used as TRPV3 modulators, and processes for the synthesis of these compounds. Pharmaceutically acceptable salts, enantiomers, and diastereomers of compounds described herein are separately and individually contemplated. Pharmaceutical compositions containing the described compounds together with pharmaceutically acceptable carriers, excipients or diluents, which can be used for the treatment of diseases, condition and/or disorders mediated by TRPV3 are separately contemplated.

The invention is defined by the claims and not limited by the description provided herein below. The terms used in the appended claims are defined herein in this glossary section, with the proviso that the claim terms may be used in a different manner if so defined by express recitation.

The terms “halogen” or “halo” means fluorine, chlorine, bromine, or iodine

The term “alkyl” refers to hydrocarbon chain consisting solely of carbon and hydrogen atoms, containing no unsaturation, have one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “C₁₋₆ alkyl” refers to an alkyl chain having 1 to 6 carbon atoms. Unless set forth or recited to the contrary, all alkyl groups described or claimed herein may be straight chain or branched, substituted or unsubstituted.

The term “alkenyl” refers to an hydrocarbon chain containing from 2 to 10 carbon atoms and including at least one carbon-carbon double bond. Non-limiting examples of alkenyl groups include ethenyl, 1-propenyl, 2-propenyl (allyl), iso-propenyl, 2-methyl-1-propenyl, 1-butenyl, and 2-butenyl. Unless set forth or recited to the contrary, all alkenyl groups described or claimed herein may be straight chain or branched, substituted or unsubstituted.

The term “alkynyl” refers to a hydrocarbon chain having at least one carbon-carbon triple bond, and having 2 to about 12 carbon atoms (with radicals having 2 to about 10 carbon atoms being preferred). Non-limiting examples of alkynyl groups include ethynyl, propynyl, and butynyl. Unless set forth or recited to the contrary, all alkynyl groups described or claimed herein may be straight chain or branched, substituted or unsubstituted.

The term “alkoxy” denotes an alkyl group attached via an oxygen linkage to the rest of the molecule. Representative examples of such groups are methoxy and ethoxy. Unless set forth or recited to the contrary, all alkoxy groups described or claimed herein may be straight chain or branched, substituted or unsubstituted.

The term “cycloalkyl” denotes a non-aromatic mono or multicyclic ring system of 3 to about 12 carbon atoms, such as cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups or sprirobicyclic groups, e.g., sprio(4,4)non-2-yl. Unless set forth or recited to the contrary, all cycloalkyl groups described or claimed herein may be substituted or unsubstituted.

The term “cycloalkylalkyl” refers to a cyclic ring-containing radical having 3 to about 8 carbon atoms directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl. Unless set forth or recited to the contrary, all cycloalkylalkyl groups described or claimed herein may be substituted or unsubstituted.

The term “cycloalkenyl” refers to a cyclic ring-containing radical having 3 to about 8 carbon atoms with at least one carbon-carbon double bond, such as cyclopropenyl, cyclobutenyl, and cyclopentenyl. Unless set forth or recited to the contrary, all cycloalkenyl groups described or claimed herein may be substituted or unsubstituted.

The term “aryl” refers to an aromatic radical having 6 to 14 carbon atoms, including monocyclic, bicyclic and tricyclic aromatic systems, such as phenyl, naphthyl, tetrahydronapthyl, indanyl, and biphenyl. Unless set forth or recited to the contrary, all aryl groups described or claimed herein may be substituted or unsubstituted.

The term “arylalkyl” refers to an aryl group as defined above directly bonded to an alkyl group as defined above, e.g., —CH₂C₆H₅ and —C₂H₄C₆H₅.

The term “heterocyclic ring” or “heterocyclyl” unless otherwise specified refers to substituted or unsubstituted non-aromatic 3 to 15 membered ring radical which consists of carbon atoms and from one to five heteroatoms selected from nitrogen, phosphorus, oxygen and sulfur. The heterocyclic ring radical may be a mono-, bi- or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; also, unless otherwise constrained by the definition the heterocyclic ring or heterocyclyl may optionally contain one or more olefinic bond(s). Examples of such heterocyclic ring radicals include, but are not limited to azepinyl, azetidinyl, benzodioxolyl, benzodioxanyl, chromanyl, dioxolanyl, dioxaphospholanyl, decahydroisoquinolyl, indanyl, indolinyl, isoindolinyl, isochromanyl, isothiazolidinyl, isoxazolidinyl, morpholinyl, oxazolinyl, oxazolidinyl, oxadiazolyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl, tetrahydropyranyl, thiazolinyl, thiazolidinyl, thiamorpholinyl, thiamorpholinyl sulfoxide and thiamorpholinyl sulfone. The heterocyclic ring radical may be attached to the main structure at any heteroatom or carbon atom that results in the creation of a stable structure.

The term “heterocyclylalkyl” refers to a heterocyclic ring radical directly bonded to an alkyl group. The heterocyclylalkyl radical may be attached to the main structure at any carbon atom in the alkyl group that results in the creation of a stable structure.

The term “heteroaryl” unless otherwise specified refers to substituted or unsubstituted 5 to 14 membered aromatic heterocyclic ring radical with one or more heteroatom(s) independently selected from N, O or S. The heteroaryl may be a mono-, bi- or tricyclic ring system. The heteroaryl ring radical may be attached to the main structure at any heteroatom or carbon atom that results in the creation of a stable structure. Examples of such heteroaryl ring radicals include, but are not limited to oxazolyl, isoxazolyl, imidazolyl, furyl, indolyl, isoindolyl, pyrrolyl, triazolyl, triazinyl, tetrazoyl, thienyl, thiazolyl, isothiazolyl, pyridyl, pyrimidinyl, pyrazinyl, pyridazinyl, benzofuranyl, benzothiazolyl, benzoxazolyl, benzimidazolyl, benzothienyl, benzopyranyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indolizinyl, acridinyl, phenazinyl and phthalazinyl.

The term “heteroarylalkyl” refers to a heteroaryl ring radical directly bonded to an alkyl group. The heteroarylalkyl radical may be attached to the main structure at any carbon atom in the alkyl group that results in the creation of a stable structure.

Unless otherwise specified, the term “substituted” as used herein refers to a group or moiety having one or more of the substituents attached to the structural skeleton of the group or moiety, including, but not limited to such substituents as hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenylalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR^{x}, —C(O)R^{x}, —C(S)R^{x}, —C(O)NR^{x}R^{y}, —C(O)ONR^{x}R^{y}, —NR^{x}CONR^{y}R^{z}, —N(R^{x})SOR^{y}, —N(R^{x})SO₂R^{y}, —(═N—N(R^{X})R^{y}), —NR^{x}C(O)OR^{y}, —NR^{x}R^{y}, —NR^{x}C(O)R^{y}, —NR^{x}C(S)R^{y}, —NR^{x}C(S)NR^{y}R^{z}, —SONR^{x}R^{y}, —SO₂NR^{x}R^{y}, —OR^{x}, —OR^{X}C(O)NR^{y}R^{Z}, —OR^{x}C(O)OR^{y}, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{y}, —R^{x}OC(O)R^{y}, —SR^{x}, —SOR^{x}, —SO₂R^{x}, and —ONO₂, wherein R^{x}, R^{y} and R^{z} are in dependently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, or substituted or unsubstituted heterocyclic ring. The substituents in the aforementioned “substituted” groups cannot be further substituted. For example, when the substituent on “substituted alkyl” is “substituted aryl”, the substituent on “substituted aryl” cannot be “substituted alkenyl”.

The term “treating” or “treatment” of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; or (c) relieving the disease, i.e., causing regression of the state, disorder or condition or at least one of its clinical or subclinical symptoms.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disorder or condition, is sufficient to cause the effect in the subject which is the purpose of the administration. The “therapeutically effective amount” will vary depending on the compound, the disease and its severity and the age, weight, physical condition and responsiveness of the subject to be treated.

The compound described in the present patent application may form salts. Non-limiting examples of pharmaceutically acceptable salts forming part of this patent application include salts derived from inorganic bases, salts of organic bases, salts of chiral bases, salts of natural amino acids and salts of non-natural amino acids. With respect to the overall compounds described by the Formula (I), the present patent application extends to these stereoisomeric forms and to mixtures thereof. To the extent prior art teaches synthesis or separation of particular stereoisomers, the different stereoisomeric forms of the present patent application may be separated from one another by the method known in the art, or a given isomer may be obtained by stereospecific or asymmetric synthesis. Tautomeric forms and mixtures of compounds described herein are also contemplated.

### Pharmaceutical Compositions

The pharmaceutical composition provided in the present invention includes at least one compound described herein and at least one pharmaceutically acceptable excipient (such as a pharmaceutically acceptable carrier or diluent). Preferably, the contemplated pharmaceutical compositions include the compound(s) described herein in an amount sufficient to inhibit TRPV3 receptor in a subject.

The subjects contemplated include, for example, a living cell and a mammal, including human mammal. The compound of the present invention may be associated with a pharmaceutically acceptable excipient (such as a carrier or a diluent) or be diluted by a carrier, or enclosed within a carrier which can be in the form of a capsule, sachet, paper or other container.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone.

The carrier or diluent may include a sustained release material, such as glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared by conventional techniques known in the art. For example, the active compound can be mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound can be adsorbed on a granular solid container, for example, in a sachet.

The pharmaceutical compositions may be in conventional forms, for example, capsules, tablets, aerosols, solutions, suspensions or products for topical application.

The route of administration may be any route which effectively transports the active compound of the invention to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment).

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Liquid formulations include, but are not limited to, syrups, emulsions, soft gelatin and sterile injectable liquids, such as aqueous or non-aqueous liquid suspensions or solutions. For parenteral application, particularly suitable are injectable solutions or suspensions formulation.

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

Suitable doses of the compounds for use in treating the diseases and disorders described herein can be determined by those skilled in the relevant art. Therapeutic doses are generally identified through a dose ranging study in humans based on preliminary evidence derived from the animal studies. Doses must be sufficient to result in a desired therepautic benefit without causing unwanted side effects. For example, the daily dosage of the TRPV3 modulator can range from about 0.1 to about 30.0 mg/kg. Mode of administration, dosage forms, suitable pharmaceutical excipients, diluents or carriers can also be well used and adjusted by those skilled in the art. All changes and modifications are envisioned within the scope of the present invention.

### Methods of Treatment

The present invention provides compounds and pharmaceutical formulations thereof that are useful in the treatment of diseases, conditions and/or disorders modulated by TRPV3. The present patent application further provides a method of treating a disease, condition and/or disorder modulated by TRPV3 in a subject in need thereof by administering to the subject a therapeutically effective amount of a compound or a pharmaceutical composition of the present invention.

Diseases, conditions, and/or disorders that are modulated by TRPV3 are believed to include, but are not limited to pain, nociceptive pain, dental pain, cardiac pain arising from an ischemic myocardium, pain due to migraine, acute pain, chronic pain, neuropathic pain, post-operative pain, pain due to neuralgia (e.g., post-herpetic neuralgia or trigeminal neuralgia), pain due to diabetic neuropathy, dental pain and cancer pain, inflammatory pain conditions (e.g. arthritis and osteoarthritis), arthralgia, neuropathies, neurodegeneration, retinopathy, neurotic skin disorder, stroke, urinary bladder hypersensitiveness, urinary incontinence, vulvodynia, gastrointestinal disorders such as irritable bowel syndrome, gastro-esophageal reflux disease, enteritis, ileitis, stomach-duodenal ulcer, inflammatory bowel disease, Crohn's disease, celiac disease, an inflammatory disease such as pancreatitis, a respiratory disorder such as allergic and non-allergic rhinitis, asthma or chronic obstructive pulmonary disease, irritation of skin, eye or mucous membrane, dermatitis, pruritic conditions such as uremic pruritus, fervescence, muscle spasms, emesis, dyskinesias, depression, Huntington's disease, memory deficits, restricted brain function, amyotrophic lateral sclerosis (ALS), dementia, arthritis, osteoarthritis, rheumatoid arthritis, diabetes, obesity, urticaria, actinic keratosis, keratocanthoma, alopecia, Meniere's disease, tinnitus, hyperacusis, anxiety disorders and benign prostate hyperplasia. Addiditional diseases, conditions and/or disorders modulated by TRPV3 is illustrated, for example in WO2007/056124; Wissenbach, U. et al, Biology of the cell (2004), 96, 47-54; Nilius, B. et al., Physiol Rev (2007), 87, 165-217; Okuhara, D. Y. et al, Expert Opinion on Therapeutic Targets (2007), 11, 391-401; Hu, H. Z. et al,

Journal of Cellular Physiology, (2006), 208, 201-212 and references cited therein, all of which are incorporated herein by reference in their entirety and for the purpose stated.

### General Methods of Preparation

The compounds described herein may be prepared by techniques known in the art. In addition, the compounds described herein may be prepared by following the reaction sequence as depicted in Schemes 1 to 4. Further, in the following schemes, where specific bases, acids, reagents, solvents, coupling agents, etc., are mentioned, it is understood that other bases, acids, reagents, solvents, coupling agents etc., known in the art may also be used and are therefore included within the present invention. Variations in reaction conditions, for example, temperature and/or duration of the reaction, which may be used as known in the art are also within the scope of the present invention. All the isomers of the compounds in described in these schemes, unless otherwise specified, are also encompassed within the scope of this invention.

2-Hydroxyacetophenone of the general formula (I) is either commercially available or can be prepared by the procedures as described in Buell, B. G. et al. J. Am. Chem. Soc. 1949, 71 (1), 1901-1905; Bergmann, R. et al. J. Med. Chem. 1990, 33, 492-504. The cinnamic acid derivative of formula (2) is commercially available or can be prepared using known approaches (Bergdahl, M. J. Org. Chem., 2007, 72, 5244-5259). Approaches for the synthesis of 4-chromenones (5) are reported in: Helquist, P. Synthesis, 2006, 3654-3660; Silva, A. M. S. et al. J. Het. Chem. 1998, 35 217-224. All the aryl boronic acids of the formula (7) used in the coupling reactions were purchased from commercial sources.

A general approach for the synthesis of compounds of the general formula (I) wherein R¹, R², R³, ‘m’ and ‘n’ are as defined above, is described in Scheme 1. 2-Hydroxy acetophenone of the general formula (I) is coupled with cinnamic acid of the formula (2) to give the cinnamic ester (3) under acid catalysis. The rearrangement of ester of general formula (3) to diene alcohol of the formula (4) is carried out using a suitable base such as sodium hydride or potassium hydroxide in a suitable solvent such as tetrahydrofuran at reflux temperature. Cyclization of compound of a general formula (4) using an acid catalyst (e.g. p-toluenesulfonic acid monohydrate) in the presence of suitable solvent (e.g. dimethyl sulfoxide) affords (E)-2-styrylchromones of a general formula (5). Further, halogenation of (E)-2-styrylchromones of general formula (5) with suitable reagent (e.g. N-bromosuccinimide (NBS), N-iodosccinimide (NIS), iodine/cerric ammonium nitrate) gives corresponding halo compound of general formula (6) (wherein X is halogen). Halo compound (6) is coupled with a suitable boronic acid of formula (7) wherein R² is preferably aryl, under Suzuki reaction conditions (catalytic Pd(0) in the presence of a base such as sodium carbonate or cesium carbonate) gives compounds represented by the general formula (I).

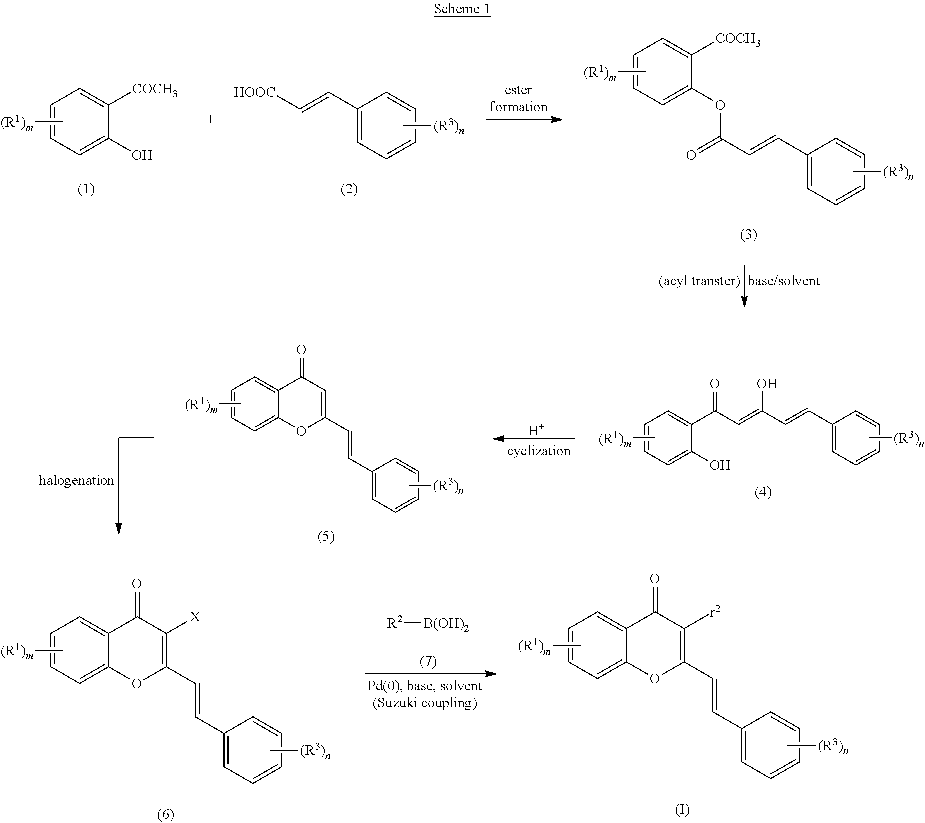

An alternative approach for the synthesis of compounds of formula (I) wherein R¹, R², R³, ‘m’ and ‘n’ are as defined above, is described in Scheme 2. 2-Hydroxyacetophenone of general formula (1) can be converted into 2-methylchromenone of general formula (8) by a method known in the literature (Brion J. D. et al. J. Het. Chem. 1991, 28, 2013-2019). Condensation of 2-methylchromones of general formula (8) with an aldehyde of a formula (9) in the presence of suitable base (example sodium hydride, sodium ethoxide, sodium methoxide) and suitable solvent (example tetrahydrofuran, ethanol, methanol) gives (E)-2-styrylchromones of general formula (5). Further, halogenation of (E)-2-styrylchromones of general formula (5) with suitable halogenating agent as described in Scheme 1 gives intermediates of the general formula (6) (wherein X is halogen). Halo compound (6) is coupled with a suitable boronic acid of formula (7) wherein R² is preferably aryl, under Suzuki reaction conditions (catalytic Pd(0) in the presence of a base such as sodium carbonate or cesium carbonate) gives compounds represented by the general formula (I).

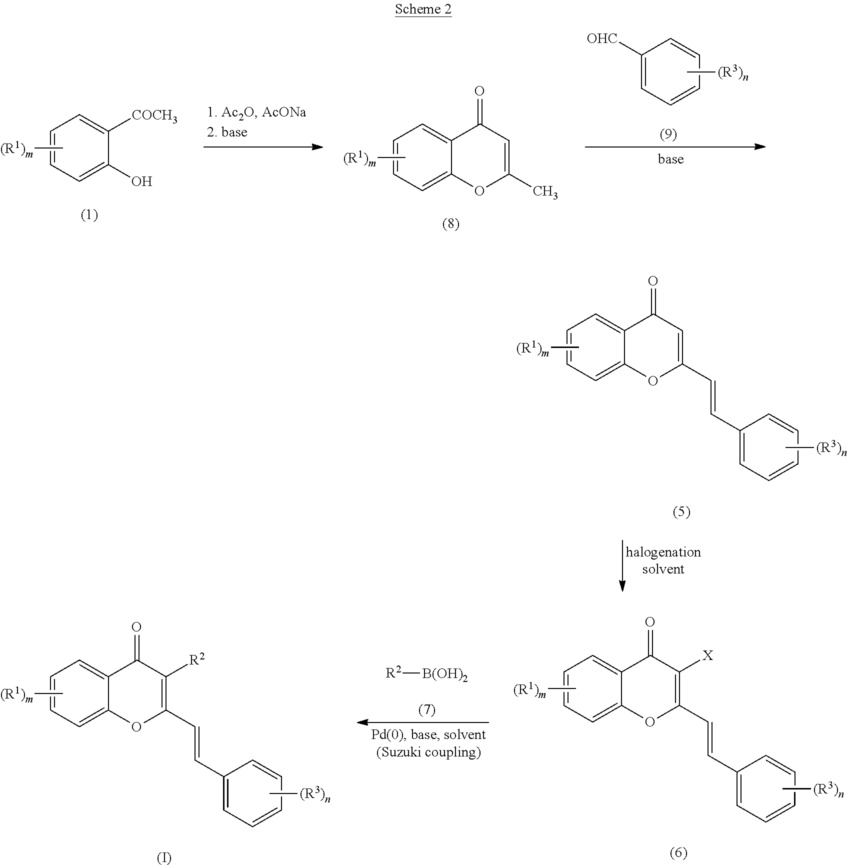

Specific compounds of the present invention represented by the general formula (II), wherein R^{a} is cyclopentyl; R¹, R^{b}, R^{c}, ‘m’ and ‘q’ are as defined above, are prepared as shown in Scheme 3. Esterification of cinnamic acid (10) with phenolic ketone of the general formula (1) followed by rearrangement of ester intermediate (11) using a suitable base such as sodium hydride in a suitable solvent (e,g. tetrahydrofuran) at reflux temperature affords intermediate of the general formula (12). Cyclization of intermediate of a general formula (12) using an appropriate acid such as p-toluenesulfonic acid in a suitable solvent such as dimethyl sulfoxide followed by halogenation of the styrylchromenone, thus formed, gives compounds of the formula (13) (wherein X is halogen). Intermediate (13) where X is preferably bromine or iodine, is coupled with a suitable aryl boronic acid of general formula (14) in the presence of a suitable Pd(0) catalyst (example Pd(PPh₃)₄) in the presence of a suitable base (example sodium carbonate) in an appropriate solvent gives compounds of the present invention represented by the general formula (II).

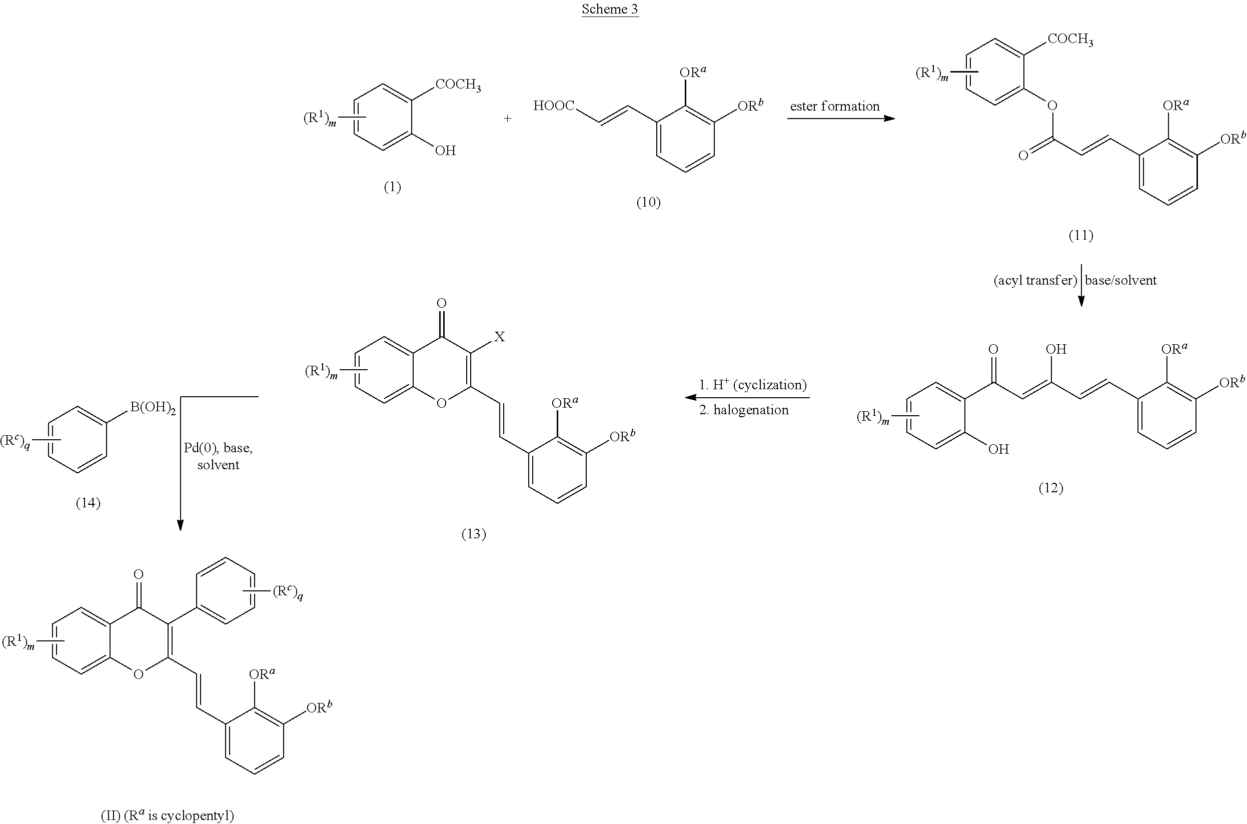

One more approach for the synthesys of specific compounds of the present invention represented by the general formula (II) is prepared as shown in Scheme 4. Selective o-decyclopentylation of compound of general formula (15), wherein R^{a} is cyclopentyl, is carried out under acidic condition (e.g. 48% hydrobromic acid in glacial acetic acid) to give corresponding monohydroxy compound of general formula (16). Alkyaltion of mono hydroxy compound of general formula (16) with appropriate alkyl halide of the formula (18) using suitable base (e.g. sodium hydride, cesium carbonate) in a suitable solvent (e.g. dimethyl sulfoxide, tetrahydrofuran) affords compound of the present invention represented by the general formula (II). Exhaustive dealkylation of compound of general formula (15) wherein R^{a} is cyclopropylmethyl, is carried out using an appropriate Lewis acid (e.g. boron tribromide) to give corresponding dihydroxy compound of general formula (17). The dihydroxy compound (17) can be alkylated in a sequential mode to give an unsymmetrical dialkoxy compound. Thus, intermediate (17) is mono alkylated using one equivalent of an appropriate electrophile of the formula (18) (wherein X is halogen) using a base such as cesium carbonate in a solvent such as tetrahydrofuran, which on further alkylation with a different electrophile of the formula (19) (wherein X is halogen) using a suitable base (e.g. sodium hydride, cesium carbonate) in a suitable solvent (e.g. dimethyl sulfoxide, tetrahydrofuran) affords compounds of the present invention represented by the general formula (II).

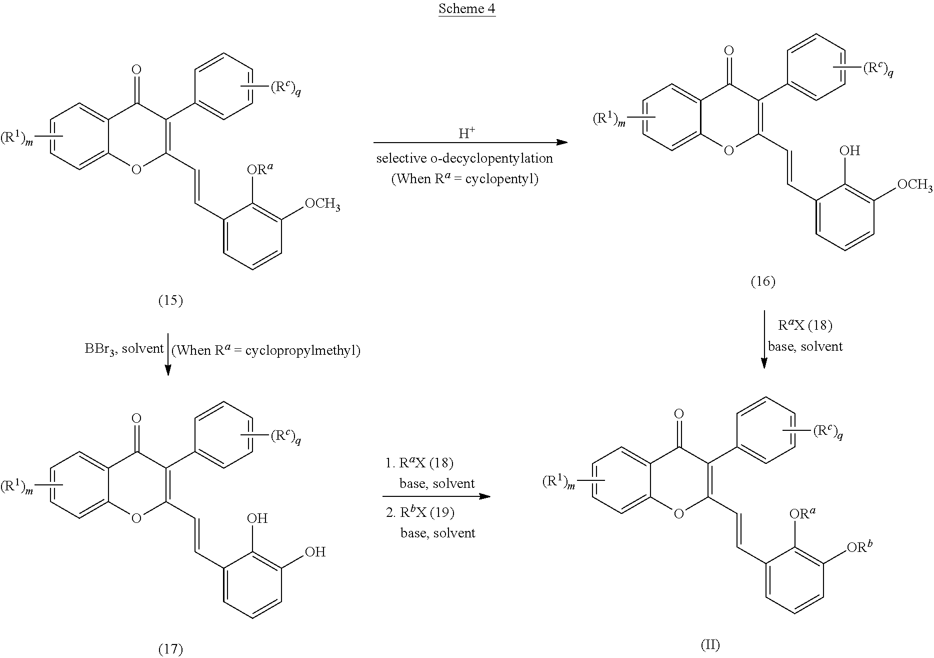

### Experimental Section

Unless otherwise stated, work-up implies the following operations: distribution of the reaction mixture between the organic and aqueous phase, separation of layers, drying the organic layer over sodium sulfate, filtration and evaporation of the organic solvent. Purification, unless otherwise mentioned, implies purification by silica gel chromatographic techniques, generally using ethyl acetate/petroleum ether mixture of a suitable polarity as the mobile phase. The following abbreviations are used in the text: DMSO-d₆: hexadeuterodimethyl sulfoxide; DMF: N,N-dimethylformamide, J: coupling constant in units of Hz; RT: room temperature (22-26° C.). aq.: aqueous; equiv.: equivalents.

### Preparation of Intermediates

All 3-iodo-4H-chromenone derivatives used for the preparation of compounds of the present invention, were prepared according to the synthetic schemes provided in ‘General Methods of Preparation’. However, these intermediates may be prepared by alternative approaches reported in the literature or by methods known to people skilled in the art of organic synthesis. Detailed experimental procedures and characterization data for the intermediates are given below.

### Intermediate 1: 2-{(E)-2-[2-(Cyclopropylmethoxy)-3-methoxyphenyl]-1-ethenyl}-3-iodo-4H-chromen-4-one

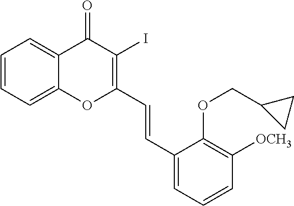

Step 1 Methyl (2E)-3-[2-(cyclopropylmethoxy)-3-methoxyphenyl]acrylate: To a stirred suspension of trimethyl phosphonoacetate (7.43 g, 21.334 mmol) in anhydrous THF (25 mL) was added sodium hydride (60% dispersion in mineral oil, 0.850 g, 21.334 mmol) at 0° C. After 30 min stirring, a solution of 2-(cyclopropylmethoxy)-3-methoxybenzaldehyde (4.0 g, 19.394 mmol) in anhydrous THF (25 mL) was added dropwise. The resulting mixture was allowed to warm to room temperature and further stirred overnight. The reaction mixture was diluted with ethyl acetate and water and layers were separated. The organic layer was washed with water (200 mL), brine (200 mL) and dried (Na₂SO₄). The organic layer was concentrated under reduced pressure and the residue obtained was purified by silica gel column chromatography using 20% ethyl acetate in petroleum ether to obtain 4.71 g of the product as colorless liquid; ¹H NMR (300 MHz, CDCl₃) δ 0.27-0.32 (m, 2H), 0.56-0.62 (m, 2H), 1.17-1.24 (m, 1H), 1.31 (t, J=7.5 Hz, 3H), 3.78-3.84 (m, 5H), 4.24 (q, J=6.9 Hz, 2H), 6.44 (d, J=15.9 Hz, 1H), 6.88-6.90 (m, 1H), 6.99-7.05 (m, 1H), 7.12-7.15 (m, 1H), 8.12 (d, J=16.2 Hz, 1H); ESI-MS (m/z) 277.17 (MH)⁺.

Step 2 (2E)-3-[2-(Cyclopropylmethoxy)-3-methoxyphenyl]acrylic acid:

To a stirred solution of Step 1 intermediate (4.60 g, 16.646 mmol) in methanol (5 mL) and THF (25 mL) was added LiOH.H₂O (1.40 g, 33.293 mmol) in water (5 mL). The mixture was stirred overnight at room temperature. Solvent was evaporated and the residue obtained was acidified with 1 N HCl to pH 4. Solid precipitated was filtered, washed with water and dried to give 3.95 g of the product as a white solid; ¹H NMR (300 MHz, CDCl₃) δ 0.26-0.31 (m, 2H), 0.56-0.62 (m, 2H), 1.22-1.29 (m, 1H), 3.83-3.85 (m, 5H), 6.48 (d, J=15.9 Hz, 1H), 6.91-6.94 (m, 1H), 7.01-7.07 (m, 1H), 7.15-7.18 (m, 1H), 8.24 (d, J=16.5 Hz, 1H); ESI-MS (m/z) 247.34 (M-H)⁻.

Step 3 2-Acetylphenyl (2E)-3-[2-(cyclopropylmethoxy)-3-methoxyphenyl]acrylate: To a stirred solution of 2′-hydroxyacetophenone (0.50 g, 3.672 mmol) in anhydrous pyridine (10 mL) was added Step 2 intermediate (1.0 g, 4.039 mmol) followed by phosphoryl chloride (1.0 mL, 11.017 mmol) at room temperature. The resulting reaction mixture was heated at 60° C. for 3 hours. The reaction mixture was poured into ice cold water and pH adjusted to 4 with 1 N hydrochloric acid. The aqueous layer was extracted with ethyl acetate (2×100 mL) and the combined organic layers were washed with water (2×100 mL), dried (Na₂SO₄) and filtered. The filtrate was concentrated under reduced pressure. The residue obtained after the evaporation of the solvent was purified by silica gel column chromatography using 2% ethyl acetate in petroleum ether to obtain 0.861 g of the product as an oil; ¹H NMR (300 MHz, CDCl₃) δ 0.28-0.31 (m, 2H), 0.55-0.60 (m, 2H), 1.23-1.28 (m, 1H), 2.57 (s, 3H), 3.86-3.88 (m, 5H), 6.70 (d, J=15.9 Hz, 1H), 6.93-6.96 (m, 1H), 7.04-7.09 (m, 1H), 7.17-7.20 (m, 2H), 7.29-7.34 (m, 1H), 7.51-7.56 (m, 1H), 7.80 (d, J=7.8 Hz, 1H), 8.34 (d, J=16.2 Hz, 1H); ESI-MS (m/z) 367.35 (MH)⁺.

Step 4 (2Z,4E)-5-[2-(Cyclopropylmethoxy)-3-methoxyphenyl]-3-hydroxy-1-(2-hydroxy-phenyl)penta-2,4-dien-1-one: To a stirred solution of Step 3 intermediate (0.85 g, 2.319 mmol) in anhydrous DMSO (5.0 mL) was added potassium hydroxide powder (0.495 g, 8.815 mmol) at room temperature under nitrogen atmosphere. After stirring for 4 h at the same temperature, the reaction mixture was poured into ice and water (100 mL) and pH adjusted to 3 by using 1 N HCl (30 mL). The hydrochloride salt precipitated out was collected by filtration. The salt was suspended in ethyl acetate (100 mL) and basified with saturated solution of NaHCO₃. The layers were separated. The aqueous layer was extracted with ethyl acetate (2×50 mL). The combined organic layers were washed with water (100 mL), brine (50 mL), filtered and evaporated under reduced pressure to afford 0.671 g of the product as yellow solid; ¹H NMR (300 MHz, CDCl₃) δ 0.28-0.32 (m, 2H), 0.58-0.64 (m, 2H), 1.21-1.28 (m, 1H), 3.83-3.85 (m, 5H), 6.29 (s, 1H), 6.64 (d, J=15.6 Hz, 1H), 6.58-6.91 (m, 3H), 6.94-7.02 (m, 1H), 7.07-7.15 (m, 1H), 7.39-7.44 (m, 1H), 7.66 (d, J=7.8 Hz, 1H), 8.06 (d, J=15.9 Hz, 1H), 12.23 (s, 1H), 14.58 (s, 1H); ESI-MS (m/z) 365.32 (M-H)⁻.

Step 5 2-{(E)-2-[2-(Cyclopropylmethoxy)-3-methoxyphenyl]-1-ethenyl}-4H-chromen-4-one: A solution of Step 4 intermediate (0.425 g, 1.159 mmol) in DMSO (5.0 mL) and p-toluenesulfonic acid monohydrate (0.110 g, 0.579 mmol) was heated at 100° C. under nitrogen atmosphere. After stirring for 3 h at the same temperature, the mixture was cooled back down to room temperature and poured into ice and water. Solid obtained was removed by filtration and dissolved in ethyl acetate (150 mL) and water (50 mL). The layers were separated. The aqueous layer was extracted with ethyl acetate (2×50 mL). The combined organic layers were washed with water (100 mL), dried (Na₂SO₄), filtered and evaporated. The crude product obtained was purified by silica gel column chromatography by using 30% ethyl acetate in petroleum ether to give 0.378 g of the product as an off-white solid; ¹H NMR (300 MHz, CDCl₃) δ 0.36-0.37 (m, 2H), 0.64-0.66 (m, 2H), 1.25-1.31 (m, 1H), 3.87-3.89 (m, 5H), 6.32 (s, 1H), 6.82-6.92 (m, 2H), 7.04-7.09 (m, 1H), 7.20-7.23 (m, 1H), 7.35-7.40 (m, 1H), 7.49-7.52 (m, 1H), 7.64-7.69 (m, 1H), 8.06 (d, J=16.2 Hz, 1H), 8.14 (d, J=7.8 Hz, 1H); ESI-MS (m/z) 349.14 (MH)⁺.

Step 6 2-{(E)-2-[2-(Cyclopropylmethoxy)-3-methoxyphenyl]-1-ethenyl}-3-iodo-4H-chromen-4-one: To a stirred solution of Step 5 Intermediate (0.245 g, 0.703 mmol) in acetonitrile (10 mL) was added cerie ammonium nitrate (0.231 g, 0.421 mmol) followed by iodine (0.124 g, 0.351 mmol) at room temperature. After stirring for 2 h at 80° C., the mixture was cooled back down to room temperature and solvent was removed under vacuum. The residue obtained was taken up in mixture of ethyl acetate (20 mL) and water (30 mL). Two layers were separated. The aqueous layer was extracted with ethyl acetate (2×50 mL). The combined organic layers were washed with water (100 mL), brine (100 mL) dried (Na₂SO₄), filtered and evaporated. The crude product obtained was purified by silica gel column chromatography by using 30% ethyl acetate in petroleum ether to give 0.121 g of the product as a light yellow solid; ¹H NMR (300 MHz, CDCl₃) δ 0.36-0.38 (m, 2H), 0.64-0.66 (m, 2H), 1.28-1.33 (m, 1H), 3.88-3.92 (m, 5H), 6.93-6.95 (m, 1H), 7.07-7.13 (m, 1H), 7.29-7.31 (m, 1H), 7.37-7.42 (m, 1H), 7.51-7.56 (m, 2H), 7.70-7.72 (m, 1H), 8.13-8.22 (m, 2H); ESI-MS (m/z) 475.91 (MH)⁺.

### Intermediate 2: 2-{(E)-2-[2-(Cyclopentyloxy)-3-methoxyphenyl]-1-ethenyl}-3-iodo-4H-chromen-4-one

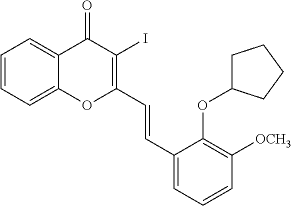

This compound was prepared in 6 steps by following the procedure described for the preparation of Intermediate 1, using 2-(cyclopentyloxy)-3-methoxybenzaldehyde and 2′-hydroxyacetophenone to give the desired product as an off-white solid; ¹H NMR (300 MHz, DMSO-d₆) δ 1.64-1.69 (m, 4H), 1.82-1.91 (m, 4H), 3.83 (s, 3H), 4.95-4.99 (m, 1H), 7.00-7.11 (m, 3H), 7.12-7.16 (m, 1H), 7.32-7.36 (m, 1H), 7.45-7.60 (m, 2H), 7.87 (t, J=8.4 Hz, 1H), 7.99-8.10 (m, 1H); ESI-MS (m/z) 489.05 (MH)⁺.

### Intermediate 3: 2-{(E)-2-[2-(Cyclopropylmethoxy)-3-methoxyphenyl]-1-ethenyl}-7-fluoro-3-iodo-4H-chromen-4-one

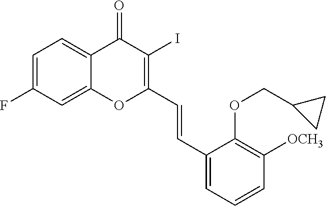

This compound was prepared in 6 steps by following the procedure described for the preparation of Intermediate 1, using 4′-fluoro-2′-hydroxyacetophenone and 2-(cyclopropylmethoxy)-3-methoxybenzaldehyde to give the desired product as an off-white solid; ¹H NMR (300 MHz, CDCl₃) δ 0.35-0.37 (m, 2H), 0.58-0.66 (m, 2H), 1.28-1.33 (m, 1H), 3.87-3.92 (m, 5H), 6.93-6.96 (m, 1H), 7.07-7.19 (m, 2H), 7.23-7.54 (m, 1H), 7.59-7.74 (m, 2H), 8.12 (d, J=16.2 Hz, 1H), 8.20-8.25 (m, 1H); ESI-MS (m/z) 493.35 (MH)⁺.

### Intermediate 4: 2-[(E)-2-(2-Cyclopentyloxy-3-methoxyphenyl)-1-ethenyl]-7-fluoro-3-iodo-4H-chromen-4-one

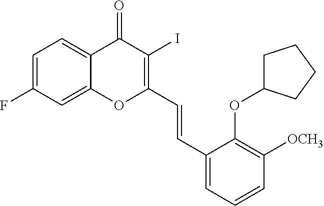

This compound was prepared in 6 steps by following the procedure described for the preparation of Intermediate 1, using 4′-fluoro-2′-hydroxyacetophenone and 2-(cyclopentyloxy)-3-methoxybenzaldehyde to give the desired product as an off-white solid; ¹H NMR (300 MHz, CDCl₃) δ 1.72-1.78 (m, 5H), 1.93-1.98 (m, 3H), 3.88 (s, 3H), 4.96 (br s, 1H), 7.12-7.20 (m, 3H), 7.59-7.66 (m, 1H), 7.72-7.81 (m, 1H), 7.89-8.00 (m, 1H), 8.24-8.30 (m, 2H); ESI-MS (m/z) 507.18 (MH)⁺.

### Intermediate 5: 2-{(E)-2-[2-(Cyclopropylmethoxy)-3-methoxyphenyl]vinyl}-6-fluoro-3-iodo-4H-chromen-4-one

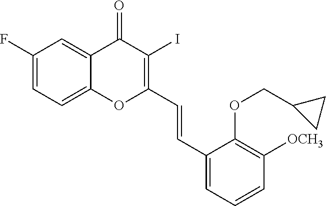

This compound was prepared in 6 steps by following the procedure described for the preparation of Intermediate 1, using 5′-fluoro-2′-hydroxyacetophenone and 2-(cyclopropylmethoxy)-3-methoxybenzaldehyde to give the desired product as an off-white solid; ¹H NMR (300 MHz, CDCl₃) δ 0.35-0.37 (m, 2H), 0.61-0.65 (m, 2H), 1.29-1.31 (m, 1H), 3.88-3.91 (m, 5H), 6.93-6.95 (m, 1H), 7.10 (t, J=7.8 Hz, 1H), 7.28-7.30 (m, 1H), 7.38-7.44 (m, 1H), 7.51-7.56 (m, 1H), 7.58 (d, J=15.6 Hz, 1H), 7.76-7.86 (m, 1H), 8.14 (d, J=16.2 Hz, 1H); ESI-MS (m/z) 493.30 (MH)⁺.

Intermediate-6: 2-[(E)-2-(2-(Cyclopentyloxy-3-methoxyphenyl)-1-ethenyl]-6-fluoro-3-iodo-4H-chromen-4-one

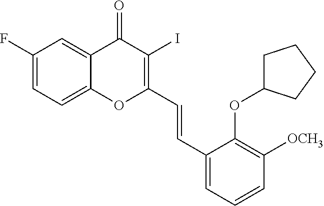

This compound was prepared in 6 steps by following the procedure described for the preparation of Intermediate 1, using 5′-fluoro-2′-hydroxyacetophenone and 2-(cyclopentyloxy)-3-methoxybenzaldehyde to give the desired product as an off-white solid; ¹H NMR (300 MHz, CDCl₃) δ 1.68-1.72 (m, 4H), 1.92-1.99 (m, 4H), 3.89 (s, 3H), 5.01 (br s, 1H), 6.97 (d, J=8.1 Hz, 1H), 7.11 (t, J=7.8 Hz, 1H), 7.33 (d, J=7.8 Hz, 1H), 7.44-7.57 (m, 3H), 7.78-7.86 (m, 2H); APCI-MS (m/z) 507.21 (MH)⁺.

### Intermediate 7: 6-Chloro-2-{(E)-2-[2-(cyclopropylmethoxy)-3-methoxyphenyl]vinyl}-3-iodo-4H-chromen-4-one

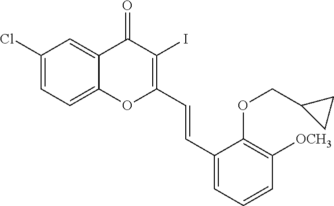

This compound was prepared in 6 steps by following the procedure described for the preparation of Intermediate 1, using 5′-chloro-2′-hydroxyacetophenone and 2-(cyclopropylmethoxy)-3-methoxybenzaldehyde to give the desired product as an off-white solid; ¹H NMR (300 MHz, CDCl₃) δ 0.35-0.38 (m, 2H), 0.61-0.67 (m, 2H), 1.25-1.33 (m, 1H), 3.88-3.91 (m, 5H), 6.93-6.96 (m, 1H), 7.10 (t, J=7.8 Hz, 1H), 7.27-7.30 (m, 1H), 7.46-7.55 (m, 2H), 7.61-7.65 (m, 2H), 8.11-8.17 (m, 1H); ESI-MS (m/z) 509.81 (MH)⁺.

### Intermediate 8: 2-[(E)-2-(2-(Cyclopropylmethoxy-3-methoxyphenyl)-1-ethenyl]-6,8-difluoro-3-iodo-4H-chromen-4-one

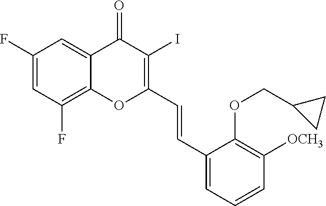

This compound was prepared in 6 steps by following the procedure described for the preparation of Intermediate 1, using 3′,5′-difluoro-2′-hydroxyacetophenone and 2-(cyclopropylmethoxy)-3-methoxybenzaldehyde to give the desired product as an off-white solid; ¹H NMR (300 MHz, CDCl₃) δ 0.34-0.40 (m, 2H), 0.61-0.66 (m, 2H), 1.24-1.31 (m, 1H), 3.79-3.91 (m, 5H), 6.94 (d, J=7.8 Hz, 1H), 7.10 (t, J=8.4 Hz, 1H), 7.29 (d, J=7.8 Hz, 1H), 7.38-7.42 (m, 1H), 7.48-7.54 (m, 1H), 7.59 (d, J=15.6 Hz, 1H), 8.14 (d, J=16.2 Hz, 1H); ESI-MS (m/z) 511.03 (MH)⁺.

### Intermediate 9: 2-{(E)-2-[2-(Cyclopentyloxy)-3-methoxyphenyl]vinyl}-6,7-difluoro-3-iodo-4H-chromen-4-one

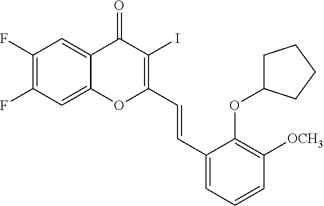

This compound was prepared in 6 steps by following the procedure described for the preparation of Intermediate 1, using 4′,5′-difluoro-2′-hydroxyacetophenone and 2-(cyclopentyloxy)-3-methoxybenzaldehyde to give the desired product as an off-white solid; ¹H NMR (300 MHz, CDCl₃) δ 1.70-1.80 (m, 4H), 1.90-1.98 (m, 4H), 3.97 (s, 3H), 5.20 (br s, 1H), 6.34 (s, 1H), 6.88 (d, J=16.2 Hz, 1H), 7.72-7.78 (m, 1H), 7.89-7.98 (m, 3H), 8.12-8.18 (m, 1H); ESI-MS (m/z) 525.08 (MH)⁺.

### EXAMPLES

The present invention is further demonstrated by preparation of the following non-limiting examples provided below. These examples are provided for illustrative purpose and not to limit the scope of the claims incorporated herein.

### Example 1

### 2-[(E)-2-(2-Cyclopropylmethoxy-3-methoxyphenyl)-1-ethenyl]-3-(4-trifluoromethoxy-phenyl)-4H-4-chromenone

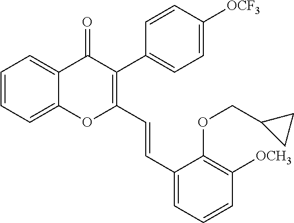

To a stirred solution of the Intermediate 1 (55 mg, 0.115 mmol) in a mixture of toluene (5.0 mL) and ethanol (2.0 mL) was added 4-(trifluoromethoxy)phenylboronic acid (33 mg, 0.162 mmol) and tetrakis(triphenylphosphine)palladium(0) (5.0 mg, 0.004 mmol) followed by sodium carbonate (74 mg, 0.695 mmol) in water (2.0 mL). The reaction mixture was refluxed for 4 h under nitrogen atmosphere. The reaction mixture was cooled to room temperature and solvent was evaporated. The residue obtained was partitioned between ethyl acetate (25 mL) and water (15 mL). The layers were separated. The aqueous layer was extracted with ethyl acetate (2×15 mL) and the combined organic layers were washed with water (2×15 mL), brine (15 ml), dried (Na₂SO₄) and filtered. The filtrate was concentrated under reduced pressure. The residue obtained after the evaporation of the solvent was purified by silica gel column chromatography using 2% ethyl acetate in petroleum ether to obtain 42 mg of the product as an off-white solid; IR (KBr) 2947, 1622, 1450, 1263, 1063, 768 cm⁻¹; ¹H NMR (300 MHz, CDCl₃) δ 0.33-0.34 (m, 2H), 0.61-0.63 (m, 2H), 1.20-1.32 (m, 1H), 3.83-3.85 (m, 5H), 6.84-6.89 (m, 2H), 6.94-7.03 (m, 2H), 7.28-7.31 (m, 2H), 7.39-7.42 (m, 3H), 7.54-7.56 (m, 1H), 7.68-7.73 (m, 1H), 8.12 (d, J=16.2 Hz, 1H), 8.22 (d, J=7.8 Hz, 1H); ESI-MS (m/z) 509.41 (MH)⁺.

### Example 2

### 2-[(E)-2-(2-Cyclopropylmethoxy-3-methoxyphenyl)-1-ethenyl]-3-(4-trifluoromethyl-phenyl)-4H-4-chromenone

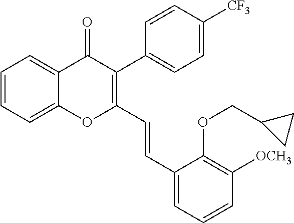

Coupling reaction of Intermediate 1 (55 mg, 0.115 mmol) with 4-(trifluoromethyl)phenyl boronic acid (31 mg, 0.162 mmol) in presence of (Ph₃P)₄Pd (5.0 mg, 0.004 mmol) according to the procedure described in Example 1, gave 34 mg of the product as a light yellow solid; IR (KBr) 3432, 2944, 1622, 1466, 1271, 1094, 768 cm⁻¹; ¹H NMR (300 MHz, CDCl₃) δ 0.32-0.34 (m, 2H), 0.60-0.63 (m, 2H), 1.17-1.19 (m, 1H), 3.82-3.85 (m, 5H), 6.82-6.89 (m, 2H), 6.93-7.03 (m, 2H), 7.38-7.43 (m, 1H), 7.49-7.57 (m, 3H), 7.70-7.72 (m, 3H), 8.14 (d, J=16.2 Hz, 1H), 8.22 (d, J=7.8 Hz, 1H); ESI-MS (m/z) 493.48 (MH)⁺.

### Example 3

### 4-{2-[(E)-2-(2-Cyclopropylmethoxy-3-methoxyphenyl)-1-ethenyl]-4-oxo-4H-3-chromenyl}benzonitrile

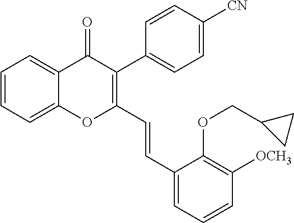

Coupling reaction of Intermediate 1 (55 mg, 0.115 mmol) with 4-cyanophenylboronic acid (21 mg, 0.149 mmol) in presence of (Ph₃P)₄Pd (5.0 mg, 0.004 mmol) according to the procedure described in Example 1, gave 30 mg of the product as a pale yellow solid; IR (KBr) 3401, 2937, 2228, 1624, 1465, 1271, 1067, 761 cm⁻¹; ¹H NMR (300 MHz, DMSO-d₆) δ 0.25-0.28 (m, 2H), 0.50-0.62 (m, 2H), 0.95-1.10 (m, 1H), 3.78-3.82 (m, 5H), 6.81 (d, J=15.6 Hz, 1H), 6.93-7.05 (m, 2H), 7.50-7.58 (m, 43H), 7.73-7.75 (m, 2H), 7.88-7.95 (m, 3H), 8.04-8.07 (m, 2H); ESI-MS (m/z) 450.27 (MH)⁺.

### Example 4

### 4-(2-{(E)-2-[2-(Cyclopropylomethoxy)-3-(difluoromethoxy)phenyl]vinyl}-4-oxo-4H-3-chromenyl}benzonitrile

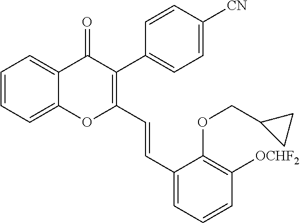

Step 1 4-}2-[(E)-2-(2,3-Dihydroxyphenyl)vinyl]-4-oxo-4H-chromen-3-yl}benzonitrile: To a well stirred and cooled (−78° C.) suspension of Example 3 (1.2 g, 2.669 mmol) in anhydrous dichloromethane (20 mL) was added solution of BBr₃ in anhydrous dichloromethane (2.006 g, 8.008 mmol) dropwise. The reaction mixture was stirred at the same temperature for 30 minutes. Then reaction mixture was warmed gradually to room temperature and stirred for 2 h. After evaporation of the solvent under reduced pressure, the reaction mixture was neutralized with saturated solution of NaHCO₃. The aqueous layer was extracted with ethyl acetate (2×100 mL) and the combined organic layers were washed with brine (2×100 mL), dried (Na₂SO₄) and filtered. The filtrate was concentrated under reduced pressure to obtain 575 mg of the product as an off-white solid; ¹H NMR (300 MHz, DMSO-d₆) δ 6.58-6.66 (m, 1H), 6.78-6.89 (m, 3H), 7.50-7.60 (m, 3H), 7.80-7.86 (m, 2H), 7.92-7.98 (m, 3H), 8.06 (d, J=7.2 Hz, 1H), 9.13 (br s, 1H), 9.68 (br s, 1H); ESI-MS (m/z) 382.20 (MH)⁺.

Step 2 4-(2-{(E)-2-[2-(Cyclopropylomethoxy)-3-hydroxyphenyl]vinyl}-4-oxo-4H-chromen-3-yl)benzonitrile: To a stirred solution of Step 1 intermediate (460 mg, 1.273 mmol) in N,N-dimethylformamide (4.0 mL) was added potassium carbonate (165 mg, 1.273 mmol) followed by (bromomethyl)cyclopropane (117 μL, 1.273 mmol) at room temperature. After stirring overnight at the same temperature, the reaction mixture was diluted with ethyl acetate (25 mL) and water (30 mL). The layers were separated. The aqueous layer was extracted with ethyl acetate (2×25 mL) and the combined organic layers were washed with water (2×25 mL), brine (25 mL), dried (Na₂SO₄) and filtered. The filtrate was concentrated under reduced pressure. The residue obtained after the evaporation of the solvent was purified by silica gel column chromatography using 20% ethyl acetate in petroleum ether to obtain 215 mg of the product as pale yellow solid; ¹H NMR (300 MHz, DMSO-d₆) δ 0.25-0.31 (m, 2H), 0.48-0.53 (m, 2H), 1.20-1.25 (m, 1H), 3.78 (d, J=6.9 Hz, 2H), 6.78-6.92 (m, 5H), 7.50-7.60 (m, 3H), 7.75 (d, J=8.1 Hz, 1H), 7.86-8.00 (m, 3H), 8.06-8.12 (m, 1H), 9.59 (br s, 1H); ESI-MS (m/z) 435.36 (MH)⁺.

Step 3 4-(2-{(E)-2-[2-(Cyclopropylomethoxy)-3-(difluoromethoxy)phenyl]vinyl}-4-oxo-4H-3-chromenyl}benzonitrile: To a stirred solution of Step 2 intermediate (172 mg, 0.834 mmol) in N,N-dimethylformamide (5.0 mL) was added cesium carbonate (54 mg, 1.668 mmol) at room temperature. The temperature of the resulting reaction mixture was raised to 60° C. and chloro(difluoro)methane (C1CHF₂) gas was passed into the reaction mixture till TLC indicated completion of the reaction. The reaction mixture was cooled to room temperature and diluted with ethyl acetate (25 mL) and water (30 mL). The layers were separated. Aqueous layer was extracted with ethyl acetate (2×25 mL) and the combined organic layers were washed with water (2×25 mL), brine (25 mL), dried (Na₂SO₄) and filtered. The filtrate was concentrated under reduced pressure. The residue obtained after the evaporation of the solvent was purified by silica gel column chromatography using 20% ethyl acetate in petroleum ether to obtain 25 mg of the product as pale yellow solid; ¹H NMR (300 MHz, DMSO-d₆) δ 0.30-0.36 (m, 2H), 0.62-0.68 (m, 2H), 1.18-1.26 (m, 1H), 3.87 (d, J=6.9 Hz, 2H), 6.56 (t, J=74.1 Hz, 1H), 6.78-6.85 (m, 1H), 7.08 (d, J=7.8 Hz, 1H), 7.17 (d, J=7.8 Hz, 1H), 7.45 (t, J=7.2 Hz, 1H), 7.47-7.58 (m, 3H), 7.72-7.82 (m, 3H), 8.11 (d, J=16.2 Hz, 1H), 8.25 (d, J=7.5 Hz, 1H); ESI-MS (m/z) 485.45 (MH)⁺.

### Example 5

### 4-{2-[(E)-2-(2-Cyclopentyloxy-3-methoxyphenyl)-1-ethenyl]-4-oxo-4H-3-chromenyl}benzonitrile

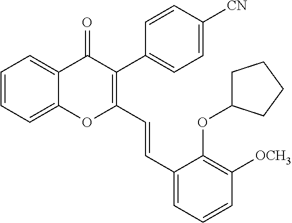

Coupling reaction of Intermediate 2 (1.00 g, 2.047 mmol) with 4-cyanophenylboronic acid (0.421 g, 2.867 mmol) in presence of (Ph₃P)₄Pd (0.094 mg, 0.081 mmol) according to the procedure described in Example 1, gave 400 mg of the product as an off-white solid; IR (KBr) 2965, 2232, 1620, 1461, 1270, 1065, 759 cm⁻¹; ¹H NMR (300 MHz, DMSO-d₆) δ 1.62-1.68 (m, 4H), 1.70-1.84 (m, 4H), 3.79 (s, 3H), 4.89-4.91 (m, 1H), 6.75 (d, J=16.2 Hz, 1H), 7.00-7.10 (m, 3H), 7.50-7.58 (m, 3H), 7.63 (d, J=7.8 Hz, 1H), 7.82-7.99 (m, 4H), 8.05 (d, J=6.3 Hz, 1H); ESI-MS (m/z) 464.16 (MH)⁺.

### Example 6

### 4-{2-[(E)-2-(2-Hydroxy-3-methoxyphenyl)-1-ethenyl]-4-oxo-4H-3-chromenyl}-benzonitrile

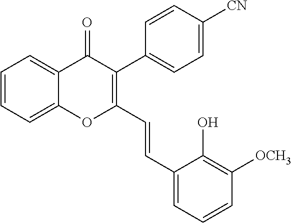

A solution of Example 5 (0.400 g, 0.863 mmol) in a mixture of 48% hydrobromic acid (10 mL) and glacial acetic acid (10 mL) was stirred at 60° C. for 2 h. The reaction mixture was neutralized with saturated solution of NaHCO₃ and extracted with ethyl acetate (2×100 mL). The combined organic layers were washed with water (2×50 mL), brine (50 mL), dried (Na₂SO₄). The filtrate was concentrated under reduced pressure. The obtained residue was purified by silica gel column chromatography to give 0.250 g of the product as an off-white solid; IR (KBr) 3429, 2227, 1620, 1466, 1262, 1088, 833 cm⁻¹; ¹H NMR (300 MHz, DMSO-d₆) δ 3.79 (s, 3H), 6.75 (t, J=7.2 Hz, 1H), 6.86 (d, J=16.2 Hz, 1H), 6.91-6.95 (m, 2H), 7.48 (t, J=7.5 Hz, 1H), 7.55 (d, J=7.8 Hz, 2H), 7.75-7.84 (m, 2H), 7.90-7.95 (m, 3H), 8.04 (d, J=7.8 Hz, 1H), 9.41 (s, 1H); ESI-MS (m/z) 396.35 (MH)⁺.

### Example 7

### 4-{2-[(E)-2-[2-(2,2-Dimethylpropoxy)-3-methoxyphenyl]-1-ethenyl]-4-oxo-4H-3-chromenyl}benzonitrile

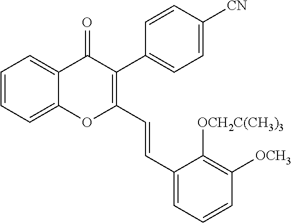

To a stirred solution of Example 6 (70 mg, 0.177 mmol) in N,N-dimethylformamide (5.0 mL) was added cesium carbonate (110 mg, 0.340 mmol) followed by 1-bromo-2,2-dimethylpropane (33.70 μL, 0.260 mmol) at room temperature. After stirring overnight at 80° C., the reaction mixture was cooled to room temperature and diluted with ethyl acetate (25 mL) and water (30 mL). The layers were separated. The aqueous layer was extracted with ethyl acetate (2×25 mL) and the combined organic layers were washed with water (2×25 mL), brine (25 mL), dried (Na₂SO₄) and filtered. The filtrate was concentrated under reduced pressure. The residue obtained after the evaporation of the solvent was purified by silica gel column chromatography using 5% ethyl acetate in petroleum ether to obtain 25 mg of the product as pale yellow solid; ¹H NMR (300 MHz, CDCl₃) δ 1.17 (s, 9H), 3.66 (s, 2H), 3.84 (s, 3H), 6.68 (d, J=16.2 Hz, 1H), 6.87-6.99 (m, 3H), 7.23-7.51 (m, 4H), 7.67-7.76 (m, 3H), 8.17-8.22 (m, 2H); ESI-MS (m/z) 466.39 (MH)⁺.

### Example 8

### 4-{2-[(E)-2-[2-Isobutoxy-3-methoxyphenyl)-1-ethenyl]-4-oxo-4H-3-chromenyl}benzonitrile

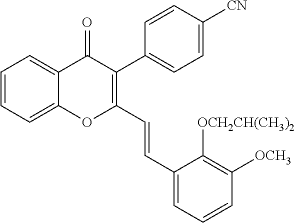

Alkylation of Example 6 (70 mg, 0.177 mmol) with 1-bromo-2-methylpropane (33 mg, 0.247 mmol) in the presence of cesium carbonate (115 mg, 0.354 mmol) in N,N-dimethylformamide (5.0 mL) according to the procedure described in Example 7, gave 35 mg of the product as pale yellow solid; IR (KBr) 3430, 2230, 1621, 1462, 1272, 1067, 836 cm⁻¹; ¹H NMR (300 MHz, DMSO-d₆) δ 1.01 (d, J=6.3 Hz, 6H), 1.80-1.90 (m, 1H), 3.67 (d, J=6.3 Hz, 2H), 3.79 (s, 3H), 6.79 (d, J=16.2 Hz, 1H), 7.04-7.08 (m, 3H), 7.49-7.57 (m, 3H), 7.63 (d, J=8.4 Hz, 1H), 7.85-7.90 (m, 1H), 7.94 (d, J=8.1 Hz, 2H), 8.01-8.05 (m, 2H); ESI-MS (m/z) 452.59 (MH)⁺.

### Example 9

### 4-(2-{(E)-2-[3-Methoxy-2-(3,3,3-trifluoropropoxy)phenyl]-1-ethenyl}-4-oxo-4H-3-chromenyl}benzonitrile

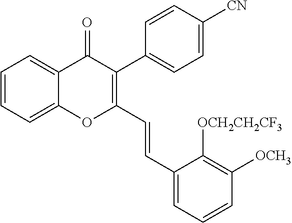

Alkylation of Example 6 (70 mg, 0.177 mmol) with 1,1,1-trifluoro-3-iodopropane (118 mg, 0.531 mmol) in the presence of cesium carbonate (115 mg, 0.354 mmol) in N,N-dimethylformamide (5.0 mL) according to the procedure described in Example 7, gave 30 mg of the product as an off-white solid; IR (KBr) 3426, 2229, 1621, 1469, 1274, 1064, 757 cm⁻¹; ¹H NMR (300 MHz, DMSO-d₆) δ 2.49-2.74 (m, 2H), 3.81 (s, 3H), 4.14 (t, J=5.4 Hz, 2H), 6.75 (d, J=16.2 Hz, 1H), 7.05-7.10 (m, 3H), 7.50 (t, J=7.5 Hz, 1H), 7.57 (d, J=8.4 Hz, 2H), 7.67 (d, J=8.4 Hz, 1H), 7.87-7.96 (m, 3H), 8.01-8.06 (m, 2H); ESI-MS (m/z) 492.35 (MH)⁺.

### Example 10

### 4-{2-[(E)-2-(2-Cyclopropylmethoxy-3-methoxyphenyl)-1-ethenyl]-7-fluoro-4-oxo-4H-3-chromenyl}benzonitrile

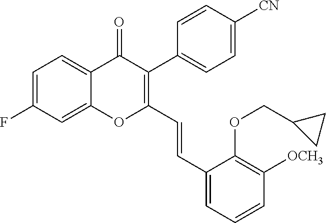

Coupling reaction of Intermediate 3 (55 mg, 0.115 mmol) with 4-cyanophenylboronic acid (33 mg, 0.162 mmol) in presence of (Ph₃P)₄Pd (5.0 mg, 0.004 mmol) according to the procedure described in Example 1, gave 42 mg of the product as pale yellow solid; IR (KBr) 2945, 2228, 1622, 1444, 1272, 1065, 785 cm⁻¹; ¹H NMR (300 MHz, CDCl₃) δ 0.31-0.33 (m, 2H), 0.59-0.63 (m, 2H), 1.15-1.20 (m, 1H), 3.83-3.85 (m, 5H), 6.76 (d, J=16.2 Hz, 1H), 6.87-6.92 (m, 2H), 6.98-7.03 (m, 1H), 7.11-7.16 (m, 1H), 7.21-7.24 (m, 1H), 7.48 (d, J=7.8 Hz, 2H), 7.75 (d, J=7.8 Hz, 2H), 8.11 (d, J=16.2 Hz, 1H), 8.20-8.25 (m, 1H); ESI-MS (m/z) 468.20 (MH)⁺.

### Example 11

### 4-{2-[(E)-2-(2-Cyclopentyloxy-3-methoxyphenyl)-1-ethenyl]-7-fluoro-4-oxo-4H-3-chromenyl}benzonitrile

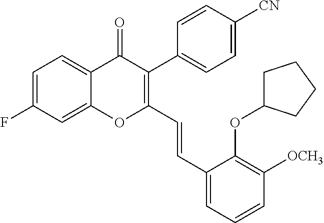

Coupling reaction of Intermediate 4 (1.70 g, 3.400 mmol) with 4-cyanophenylboronic acid (0.556 g, 3.800 mmol) in presence of (Ph₃P)₄Pd (0.157 g, 0.13 mmol) according to the general procedure described in Example 1, gave 0.600 g of the product as an off-white solid; IR (KBr) 3444, 2963, 2223, 1618, 1441, 1267, 1066, 779 cm⁻¹; ¹H NMR (300 MHz, CDCl₃) δ 1.65-1.72 (m, 4H), 1.85-1.93 (m, 4H), 3.87 (s, 3H), 4.97 (br s, 1H), 6.73 (d, J=16.2 Hz, 1H), 6.93-6.98 (m, 2H), 7.12-7.22 (m, 2H), 7.51 (d, J=7.8 Hz, 1H), 7.69 (d, J=8.4 Hz, 2H), 7.78 (d, J=6.9 Hz, 2H), 8.06 (d, J=15.9 Hz, 1H), 8.22-8.28 (m, 1H); APCI-MS (m/z) 482.24 (MH)⁺.

### Example 12

### 4-{7-Fluoro-2-[(E)-2-(3-methoxy-2-neopentyloxyphenyl)-1-ethenyl]-4-oxo-4H-3-chromenyl}benzonitrile

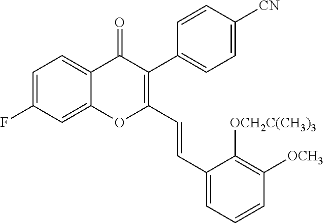

Step 1 4-{7-Fluoro-2-[(E)-2-(2-hydroxy-3-methoxyphenyl)-1-ethenyl]-4-oxo-4H-3-chromenyl}benzonitrile: Decyclopentylation of Example 11 (0.275 g, 0.572 mmol) with 48% hydrobromic acid (10 mL) in glacial acetic acid (10 mL) according to the procedure described in Example 6 gave 0.150 g of the product as pale yellow solid; ¹H NMR (300 MHz, CDCl₃) δ 3.92 (s, 3H), 6.15 (s, 1H), 6.80-6.86 (m, 2H), 6.90-6.96 (m, 2H), 7.15 (t, J=7.2 Hz, 1H), 7.31 (d, J=9.3 Hz, 1H), 7.52 (d, J=7.8 Hz, 2H), 7.77 (d, J=7.8 Hz, 2H), 7.91 (d, J=16.2 Hz, 1H), 8.24 (t, J=8.4 Hz, 1H); APCI-MS (m/z) 414.38 (MH)⁺.

Step 2 4-{7-Fluoro-2-[(E)-2-(3-methoxy-2-neopentyloxyphenyl)-1-ethenyl]-4-oxo-4H-3-chromenyl}benzonitrile: Step 1 intermediate (70 mg, 0.160 mmol) was alkylated with 1-bromo-2,2-dimethylpropane (64.5 μL, 0.500 mmol) in the presence of cesium carbonate (104 mg, 0.320 mmol) according to the procedure described in Example 7 to afford 30 mg of the product as pale yellow solid; IR (KBr) 3443, 2230, 1619, 1440, 1272, 1067, 842 cm⁻¹; ¹H NMR (300 MHz, CDCl₃) δ 1.17 (s, 9H), 3.67 (s, 2H), 3.86 (s, 3H), 6.69 (d, J=15.6 Hz, 1H), 6.93-7.00 (m, 3H), 7.13 (d, J=8.7 Hz, 2H), 7.52 (d, J=7.8 Hz, 2H), 7.78 (d, J=7.2 Hz, 2H), 8.15-8.27 (m, 2H); APCI-MS (m/z) 484.22 (MH)⁺.

### Example 13

### chromenyl}benzonitrile

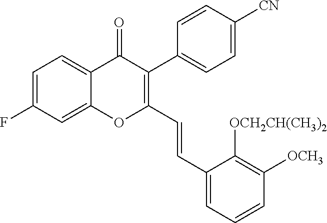

Alkylation of Step 1 intermediate of Example 12 (65 mg, 0.150 mmol) with 1-bromo-2-methylpropane (51.33 μL, 0.470 mmol) in the presence of cesium carbonate (97 mg, 0.300 mmol) in N,N-dimethylformamide (5.0 mL) according to the procedure described in Example 7, gave 32 mg of the product as pale yellow solid; IR (KBr) 3430, 2230, 1621, 1462, 1272, 1067, 836 cm⁻¹; ¹H NMR (300 MHz, CDCl₃) δ 1.13 (d, J=6.6 Hz, 6H), 2.04-2.13 (m, 1H), 3.76 (d, J=6.9 Hz, 2H), 3.87 (s, 3H), 6.75 (d, J=16.2 Hz, 1H), 6.92 (d, J=6.3 Hz, 2H), 7.03 (d, J=8.1 Hz, 1H), 7.18 (d, J=9.3 Hz, 2H), 7.51 (d, J=8.1 Hz, 2H), 7.77 (d, J=8.4 Hz, 2H), 8.12 (d, J=15.9 Hz, 1H), 8.25 (t, J=8.4 Hz, 1H); APCI-MS (m/z) 470.20 (MH)⁺.

### Example 14

### 4-(7-Fluoro-2-{(E)-2-[3-methoxy-2-(3,3,3-trifluoropropoxy)phenyl]-1-ethenyl}-4-oxo-4H-3-chromenyl)benzonitrile

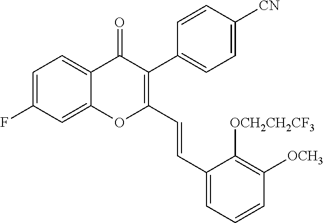

Alkylation of Step 1 intermediate of Example 12 (90 mg, 0.210 mmol) with 1,1,1-trifluoro-3-iodopropane (128 μL, 1.00 mmol) in the presence of cesium carbonate (136 mg, 0.420 mmol) in N,N-dimethylformamide (5.0 mL) according to the procedure described in Example 7, gave 28 mg of the product as an off-white solid; IR (KBr) 3426, 2229, 1621, 1469, 1274, 1064, 757 cm⁻¹; ¹H NMR (300 MHz, CDCl₃) δ 2.57-2.66 (m, 2H), 3.88 (s, 3H), 4.26 (t, J=6.0 Hz, 2H), 6.73 (d, J=15.6 Hz, 1H), 6.90-6.98 (m, 2H), 7.05 (d, J=7.8 Hz, 1H), 7.16 (t, J=8.1 Hz, 1H), 7.28-7.34 (m, 1H), 7.51 (d, J=7.8 Hz, 2H), 7.79 (d, J=7.8 Hz, 2H), 8.07 (d, J=15.9 Hz, 1H), 8.25 (t, J=7.8 Hz, 1H); APCI-MS (m/z) 510.22 (MH)⁺.

### Example 15

### 4-{2-[(E)-2-(2-Cyclopropylmethoxy-3-methoxyphenyl)-1-ethenyl]-6-fluoro-4-oxo-4H-3-chromenyl}benzonitrile

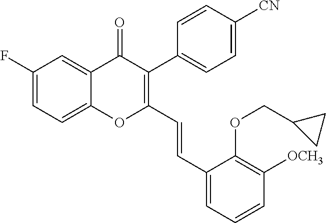

Coupling reaction of Intermediate 5 (80 mg, 0.162 mmol) with 4-cyanophenylboronic acid (26 mg, 0.176 mmol) in presence of (Ph₃P)₄Pd (7.0 mg, 0.006 mmol) according to the procedure described in Example 1, gave 56 mg of the product as pale yellow solid; IR (KBr) 3066, 2229, 1624, 1482, 1271, 1069, 747 cm⁻¹; ¹H NMR (300 MHz, CDCl₃) δ 0.25-0.26 (m, 2H), 0.50-0.53 (m, 2H), 0.98-1.12 (m, 1H), 3.73-3.78 (m, 5H), 6.81 (d, J=16.2 Hz, 1H), 7.56 (d, J=8.4 Hz, 2H), 7.71-7.87 (m, 3H), 7.94 (d, J=8.1 Hz, 2H), 8.03 (d, J=15.9 Hz, 1H); ESI-MS (m/z) 468.24 (MH)⁺.

### Example 16

### 4-{2-[(E)-2-(2-Cyclopentyloxy-3-methoxyphenyl)-1-ethenyl]-6-fluoro-4-oxo-4H-3-chromenyl}benzonitrile

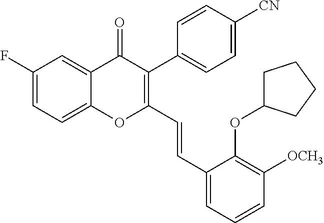

Coupling reaction of Intermediate 6 (4.50 g, 8.887 mmol) with 4-cyanophenylboronic acid (1.436 g, 9.776 mmol) in presence of (Ph₃P)₄Pd (41 mg, 0.355 mmol) according to the procedure described in Example 1, gave 1.70 g of the product as an off-white solid; IR (KBr) 3444, 2960, 2233, 1625, 1480, 1263, 1062, 746 cm⁻¹; δ ¹H NMR (300 MHz, CDCl₃) δ 1.64-1.70 (m, 4H), 1.80-1.88 (m, 4H), 3.87 (s, 3H), 4.97 (br s, 1H), 6.74 (d, J=16.2 Hz, 1H), 6.92-7.01 (m, 2H), 7.45-7.55 (m, 5H), 7.78 (d, J=8.1 Hz, 2H), 7.82-7.88 (m, 1H), 8.09 (d, J=16.2 Hz, 1H); ESI-MS (m/z) 482.27 (MH)⁺.

### Example 17

### 4-{2-[(E)-2-(2-Cyclobutylmethoxy-3-methoxyphenyl)-1-ethenyl]-6-fluoro-4-oxo-4H-3-chromenyl}benzonitrile

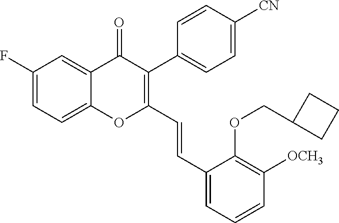

Step 1 4-{6-Fluoro-2-[(E)-2-(2-hydroxy-3-methoxyphenyl)-1-ethenyl]-4-oxo-4H-3-chromenyl}benzonitrile: Decyclopentylation of Example 16 (700 mg, 1.453 mmol) with 48% hydrobromic acid (10 mL) in glacial acetic acid (10 mL) according to the procedure described in Example 6 gave 350 mg of the product as pale yellow solid; ¹H NMR (300 MHz, DMSO-d₆) δ 3.80 (s, 3H), 6.80 (t, J=7.8 Hz, 1H), 6.87 (s, 1H), 6.92-7.00 (m, 2H), 7.58 (d, J=7.8 Hz, 2H), 7.70-7.80 (m, 2H), 7.92-7.98 (m, 4H), 9.46 (br s, 1H, exchangeable with D₂O); APCI-MS (m/z) 414.23 (MH)⁺.

Step 2 4-{2-[(E)-2-(2-Cyclobutylmethoxy-3-methoxyphenyl)-1-ethenyl]-6-fluoro-4-oxo-4H-3-chromenyl}benzonitrile: Step 1 intermediate (100 mg, 0.241 mmol) was alkylated with (bromomethyl)cyclobutane (50 mg, 0.338 mmol) in the presence of cesium carbonate (157 mg, 0.483 mmol) according to the procedure described in Example 7 to afford 35 mg of the product as pale yellow solid; IR (KBr) 3443, 2227, 1626, 1478, 1271, 999 cm⁻¹; ¹H NMR (300 MHz, CDCl₃) δ 1.91-1.96 (m, 4H), 2.15-2.21 (m, 2H), 2.69-2.74 (m, 1H), 3.87 (s, 3H), 4.00 (d, J=6.9 Hz, 2H), 6.77 (d, J=16.2 Hz, 1H), 6.89-6.96 (m, 2H), 6.94-7.01 (m, 1H), 7.42-7.54 (m, 4H), 7.79 (d, J=7.8 Hz, 2H), 7.87 (d, J=5.4 Hz, 1H), 8.10 (d, J=16.2 Hz, 1H); APCI-MS (m/z) 482.16 (MH)⁺.

### Example 18

### 4-{6-Fluoro-2-[(E)-2-(2-isopentyloxy-3-methoxyphenyl)-1-ethenyl]-4-oxo-4H-3-chromenyl}benzonitrile

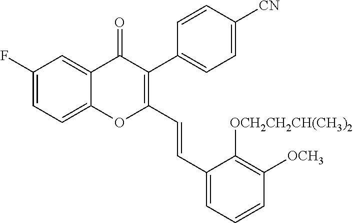

Alkylation of Step 1 intermediate of Example 17 (35 mg, 0.084 mmol) with 1-bromo-3-methylbutane (30 μL, 0.253 mmol) in the presence of cesium carbonate (53 mg, 0.253 mmol) in N,N-dimethylformamide (5.0 mL) according to the procedure described in Example 7, gave 25 mg of the product as pale yellow solid; IR (KBr) 3433, 2223, 1625, 1481, 1267, 1063, 796 cm⁻¹; ¹H NMR (300 MHz, CDCl₃) δ 0.99 (d, J=6.3 Hz, 6H), 1.64-1.71 (m, 2H), 1.90-1.96 (m, 1H), 3.87 (s, 3H), 4.02 (t, J=6.9 Hz, 2H), 6.81 (d, J=15.6 Hz, 1H), 6.92-7.00 (m, 2H), 7.04 (d, J=7.8 Hz, 1H), 7.43-7.59 (m, 4H), 7.77 (d, J=7.8 Hz, 2H), 7.88 (dd, J=3.0, 7.8 Hz, 1H), 8.05 (d, J=15.9 Hz, 1H); ESI-MS (m/z) 484.37 (MH)⁺.

### Example 19

### 4-{6-Fluoro-2-[(E)-2-(2-isobutoxy-3-methoxyphenyl)-1-ethenyl]-4-oxo-4H-3-chromenyl}benzonitrile

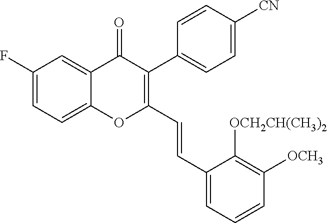

Alkylation of Step 1 intermediate of Example 17 (35 mg, 0.084 mmol) with 1-bromo-2-methylpropane (35 mg, 0.253 mmol) in the presence of cesium carbonate (83 mg, 0.253 mmol) in N,N-dimethylformamide (5.0 mL) according to the procedure described in Example 7 gave 23 mg of the product as pale yellow solid; IR (KBr) 3421, 2228, 1625, 1479, 1271, 1064, 831 cm⁻¹; ¹H NMR (300 MHz, CDCl₃) δ 1.12 (d, J=6.9 Hz, 6H), 2.04-2.10 (m, 1H), 3.77 (d, J=6.3 Hz, 2H), 3.86 (s, 3H), 6.76 (d, J=15.9 Hz, 1H), 6.91-6.99 (m, 2H), 7.03 (d, J=7.8 Hz, 1H), 7.45-7.53 (m, 4H), 7.78 (d, J=7.8 Hz, 2H), 7.87 (d, J=5.4 Hz, 1H), 8.14 (d, J=16.2 Hz, 1H); ESI-MS (m/z) 470.32 (MH)⁺.

### Example 20

### 4-{6-Fluoro-2-[(E)-2-(2,2-dimethylpropoxy)-3-methoxyphenyl]-1-ethenyl]-4-oxo-4H-3-chromenyl}benzonitrile

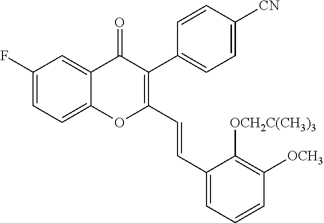

Alkylation of Step 1 intermediate of Example 17 (70 mg, 0.169 mmol) with 1-bromo-2,2-dimethylpropane (76 mg, 0.507 mmol) in the presence of cesium carbonate (165 mg, 0.507 mmol) in N,N-dimethylformamide (5.0 mL) according to the procedure described in Example 7 gave 49 mg of the product as pale yellow solid; IR (KBr) 3430, 2228, 1625, 1479, 1273, 1063, 746 cm⁻¹; ¹H NMR (300 MHz, CDCl₃) δ 1.16 (s, 9H), 3.67 (s, 2H), 3.86 (s, 3H), 6.70 (d, J=15.9 Hz, 1H), 6.90-7.04 (m, 3H), 7.40-7.48 (m, 2H), 7.52 (d, J=8.4 Hz, 2H), 7.80 (d, J=8.1 Hz, 2H), 7.87 (dd, J=2.4, 7.2 Hz, 1H), 8.22 (d, J=15.9 Hz, 1H); ESI-MS (m/z) 484.52 (MH)⁺.

### Example 21

### 4-{6-Chloro-2-[(E)-2-(2-cyclopropylmethoxy-3-methoxyphenyl)-1-ethenyl]-4-oxo-4H-3-chromenyl}benzonitrile

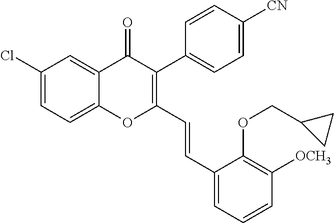

Coupling reaction of Intermediate 7 (95 mg, 0.186 mmol) with 4-cyanophenylboronic acid (38 mg, 0.261 mmol) in presence of (Ph₃P)₄Pd (9.0 mg, 0.007 mmol) according to the procedure described in Example 1, gave 35 mg of the product as a pale yellow solid; IR (KBr) 2924, 2226, 1624, 1436, 1270, 1064, 737 cm⁻¹; ¹H NMR (300 MHz, CDCl₃) δ 0.31-0.35 (m, 2H), 0.58-0.64 (m, 2H), 1.15-1.20 (m, 1H), 3.83-3.85 (m, 5H), 6.77 (d, J=16.2 Hz, 1H), 6.88-6.92 (m, 2H), 6.98-7.03 (m, 1H), 7.47-7.53 (m, 3H), 7.63-7.66 (m, 1H), 7.75 (d, J=8.4 Hz, 2H), 8.10-8.17 (m, 2H); ESI-MS (m/z) 484.39 (MH)⁺.

### Example 22

### 4-{2-[(E)-2-(2-Cyclopropylmethoxy-3-methoxyphenyl)-1-ethenyl]-6,8-difluoro-4-oxo-4H-3-chromenyl}benzonitrile

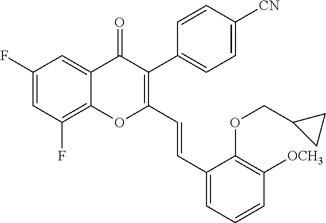

Coupling reaction of Intermediate 8 (70 mg, 0.130 mmol) with 4-cyanophenylboronic acid (22 mg, 0.150 mmol) in presence of (Ph₃P)₄Pd (6.0 mg, 0.005 mmol) according to the procedure described in Example 1, gave 20 mg of the product as an off-white solid; ¹H NMR (300 MHz, CDCl₃) δ 0.25-0.31 (m, 2H), 0.58-0.63 (m, 2H), 1.18-1.24 (m, 1H), 3.85 (d, J=7.5 Hz, 2H), 3.92 (s, 3H), 6.16 (s, 1H), 7.00 (d, J=6.3 Hz, 2H), 7.15-7.26 (m, 4H), 7.42 (d, J=8.1 Hz, 1H), 7.50-7.63 (m, 1H), 7.69 (d, J=8.4 Hz, 2H); ESI-MS (m/z) 486.47 (MH)⁺.

### Example 23

### 4-{2-[(E)-2-[2-Cyclopentyloxy-3-methoxyphenyl)-1-ethenyl]-6,7-difluoro-4-oxo-4H-3-chromenyl}benzonitrile

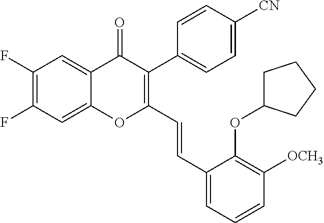

Coupling reaction of Intermediate 9 (0.90 g, 1.723 mmol) with 4-cyanophenylboronic acid (278 mg, 01.895 mmol) in presence of (Ph₃P)₄Pd (790 mg, 0.068 mmol) according to the procedure described in Example 1, gave 42 mg of the product as an off-white solid; ¹H NMR (300 MHz, CDCl₃) δ 1.66-1.72 (m, 4H), 1.85-1.92 (m, 4H), 3.87 (s, 3H), 4.97 (br s, 1H), 6.72 (d, J=16.8 Hz, 1H), 6.90-6.96 (m, 2H), 6.99 (d, J=7.8 Hz, 1H), 7.30-7.37 (m, 1H), 7.50 (d, J=7.8 Hz, 2H), 7.79 (d, J=8.4 Hz, 2H), 7.98-8.08 (m, 2H); APCI-MS (m/z) 500.37 (MH)⁺.

The preceding examples can be repeated with similar success by substituting the generically or specifically described reactants and/or operating conditions of this invention for those used in the preceding examples. While the invention has been illustrated with respect to the production and of particular compounds, it is apparent that variations and modifications of the invention can be made without departing from the spirit or scope of the invention. Upon further study of the specification, further aspects, objects and advantages of this invention will become apparent to those skilled in the art.

### Pharmacological Activity

The illustrative examples of the present invention are screened for TRPV3 activity according to a modified procedure described in (a) Tóth, A. et al. Life Sciences 2003, 73, 487-498. (b) McNamara C, R. et al. Proc. Natl. Acad. Sci. U.S.A., 2007, 104, 13525-13530. The screening of the compounds can be carried out by other methods and procedures known to persons skilled in the art.

Screening for TRPV3 Antagonist Using the ⁴⁵Calcium Uptake Assay

The inhibition of TRPV3 receptor activation was followed as inhibition of 2-aminoethxydiphenylborate (2-APB) induced cellular uptake of radioactive calcium. Test compounds were dissolved in dimethyl sulfoxide (DMSO) to prepare 20 mM stock solution and then diluted using plain medium with DMEM/F-12 containing 1.8 mM CaCl₂ to get desired concentration. Final concentration of DMSO in the reaction was 0.5% (v/v). Human TRPV3 expressing CHO cells were grown in DMEM/F-12 medium with 10% FBS, 1% penicillin-streptomycin solution, 400 μg/mL of G-418. Cells were seeded 24 h prior to the assay in 96 well plates so as to get ˜50,000 cells per well on the day of experiment. Cells were treated with test compounds for 10 minutes followed by addition of 2-APB at a final concentration of 500 μM and 5 μCi/mL ⁴⁵Ca⁺² for 4 minutes. Cells were washed and lysed using buffer containing 1% Triton X-100, 0.1% deoxycholate and 0.1% SDS. Radioactivity in the lysate was measured in Packardt Top count after addition of liquid scintillant. Concentration response curves were plotted as a % of maximal response obtained in the absence of test antagonist. IC₅₀ value was calculated from concentration response curve by nonlinear regression analysis using GraphPad PRISM software.

The compounds prepared were tested using the above assay procedure and the results obtained are given in Table 1. Percentage inhibition at concentrations of 1.0 μM and 10.0 μM are given in the table along with IC₅₀ (nM) values for selected examples.

The IC₅₀ (nM) values of the compounds are set forth in Table 1 wherein “A” refers to an IC₅₀ value of less than 50 nM, “B” refers to IC₅₀ value in range of 50.01 to 150.0 nM and “C” refers to an IC₅₀ value in range of 150.01 to 1000.0 nM.

TABLE 1
In-vitro screening results of compounds of invention

Percentage inhibition

| Examples | at 1.0 μM | at 10.0 μM | IC₅₀ (nM) |
|---|---|---|---|
| Example 1 | 36.73 | 62.19 | — |
| Example 2 | 63.11 | 75.36 | C |
| Example 3 | 90.37 | 94.46 | C |
| Example 4 | 92.94 | 94.85 | C |
| Example 5 | 71.76 | 90.29 | C |
| Example 6 | 32.10 | 83.93 | — |
| Example 7 | 89.79 | 93.24 | B |
| Example 8 | 89.27 | 96.36 | B |
| Example 9 | 94.48 | 98.05 | B |
| Example 10 | 87.34 | 95.59 | C |
| Example 11 | 84.56 | 92.61 | B |
| Example 12 | 82.19 | 91.15 | A |
| Example 13 | 92.87 | 96.71 | B |
| Example 14 | 95.99 | 98.70 | C |
| Example 15 | 79.27 | 94.12 | C |
| Example 16 | 59.43 | 74.39 | — |
| Example 17 | 78.25 | 89.83 | C |
| Example 18 | 55.19 | 71.69 | — |
| Example 19 | 70.23 | 87.81 | A |
| Example 20 | 74.34 | 78.55 | A |
| Example 21 | 76.43 | 87.69 | C |
| Example 22 | 39.95 | 72.95 | — |
| Example 23 | 75.85 | 86.66 | A |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

All publications, patents, and patent applications cited in this application are herein incorporated by reference to the same extent as if each individual publication, patent, or patent application was specifically and individually indicated to be incorporated herein by reference.

1.-29. (canceled)

30. A compound of the formula (I): 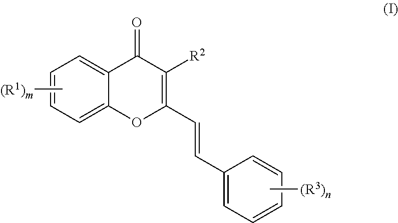
wherein,
at each occurrence, R¹ is independently selected from nitro, cyano, halogen, —OR^{a}, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heterocyclic group, —NR⁴R⁵, —S(O)ₚNR⁴R⁵, and —S(O)ₚR⁴;
R² is selected from halogen, substituted or unsubstituted aryl, substituted or unsubstituted heteroaryl, or substituted or unsubstituted heterocyclic group; wherein substituent(s) are independently selected from halogen, nitro, cyano, —NR⁴R⁵, substituted or unsubstituted alkyl, substituted or unsubstituted alkenyl, substituted or unsubstituted alkoxy, substituted or unsubstituted haloalkyl, substituted or unsubstituted haloalkyloxy, substituted or unsubstituted cycloalkyl, substituted or unsubstituted aryl, substituted or unsubstituted heterocyclic group, and substituted or unsubstituted heteroaryl;
at each occurrence, R³ may be same or different and is selected from nitro, cyano, halogen, —OR^{a}, substituted or unsubstituted alkyl, substituted or unsubstituted alkenyl, substituted or unsubstituted haloalkyl, substituted or unsubstituted cyanoalkyl, substituted or unsubstituted cyanoalkyloxy, substituted or unsubstituted cycloalkyl, substituted or unsubstituted aryl, substituted or unsubstituted heterocyclic group, and substituted or unsubstituted heteroaryl;
at each occurrence, R^{a} is independently selected from hydrogen, substituted or unsubstituted alkyl, linear or branched chain alkyl, substituted or unsubstituted haloalkyl, substituted or unsubstituted cyanoalkyl, substituted or unsubstituted alkenyl, substituted or unsubstituted cycloalkyl, substituted or unsubstituted alkoxyalkyl, substituted or unsubstituted aryl, substituted or unsubstituted heteroaryl, substituted or unsubstituted heterocyclic group, substituted or unsubstituted cycloalkylalkyl, substituted or unsubstituted arylalkyl, substituted or unsubstituted heteroarylalkyl, and substituted or unsubstituted heterocyclylalkyl;
at each occurrence, R⁴ and R⁵ are independently selected from hydrogen, substituted or unsubstituted alkyl, substituted or unsubstituted alkenyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic group, and substituted or unsubstituted heterocyclylalkyl;
‘n’ is an integer selected from 0 to 5, both inclusive;
‘m’ is an integer selected from 0 to 4, both inclusive; and
at each occurrence, ‘p’ is an integer selected from 0 to 2, both inclusive;
or pharmaceutically acceptable salt there of.

31. The compound of claim 30 having the formula (II): 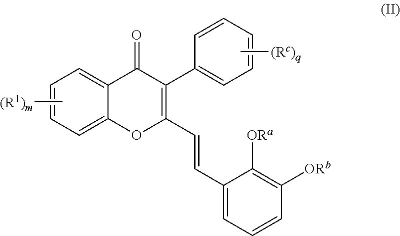
wherein,
at each occurrence, R¹ is independently selected from nitro, cyano, halogen, —OR^{a}, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroaryl, substituted or unsubstituted heterocyclic group, —NR⁴R⁵, —S(O)ₚNR⁴R⁵, and —S(O)ₚR⁴;
R^{a} is selected from hydrogen, substituted or unsubstituted alkyl, linear or branched chain alkyl, substituted or unsubstituted haloalkyl, substituted or unsubstituted cyanoalkyl, substituted or unsubstituted alkenyl, substituted or unsubstituted cycloalkyl, substituted or unsubstituted alkoxyalkyl, substituted or unsubstituted aryl, substituted or unsubstituted heteroaryl, substituted or unsubstituted heterocyclic group, substituted or unsubstituted cycloalkylalkyl, substituted or unsubstituted arylalkyl, substituted or unsubstituted heteroarylalkyl, and substituted or unsubstituted heterocyclylalkyl;
R^{b} is selected from hydrogen, substituted or unsubstituted alkyl, substituted or unsubstituted alkenyl, substituted or unsubstituted haloalkyl, substituted or unsubstituted cycloalkyl, substituted or unsubstituted aryl, substituted or unsubstituted heterocyclic group, and substituted or unsubstituted heteroaryl;
at each occurrence, R^{c} is independently selected from hydrogen, nitro, cyano, halogen, —OR^{a}, substituted or unsubstituted alkyl, substituted or unsubstituted haloalkyl, substituted or unsubstituted alkenyl, substituted or unsubstituted cycloalkyl, substituted or unsubstituted aryl, substituted or unsubstituted heteroaryl, substituted or unsubstituted heterocyclic group and —NR⁴R⁵;
at each occurrence, R⁴ and R⁵ are independently selected from hydrogen, substituted or unsubstituted alkyl, substituted or unsubstituted alkenyl, substituted or unsubstituted cycloalkyl, substituted or unsubstituted cycloalkylalkyl, substituted or unsubstituted cycloalkenyl, substituted or unsubstituted aryl, substituted or unsubstituted arylalkyl, substituted or unsubstituted heteroaryl, substituted or unsubstituted heteroarylalkyl, substituted or unsubstituted heterocyclic group, and substituted or unsubstituted heterocyclylalkyl;
‘m’ is an integer selected from 0 to 4, both inclusive;
at each occurrence, ‘p’ is an integer selected from 0 to 2, both inclusive; and
‘q’ is an integer selected from 0 to 5, both inclusive;
or pharmaceutically acceptable salt thereof.

32. The compound of claim 30, wherein each of R¹ is halogen.

33. The compound of claim 30, wherein R² is substituted aryl.

34. The compound of claim 33, wherein substituent(s) on aryl is halogen, cyano, haloalkyl, or haloalkoxy.

35. The compound of claim 30, wherein R³ is —OR^{a}.

36. The compound of claim 35, wherein R^{a} hydrogen, substituted or unsubstituted alkyl, haloalkyl, cycloalkyl, or cycloalkylalkyl.

37. The compound of claim 30, wherein ‘n’ is 2.

38. The compound of claim 30, wherein ‘m’ is 1 or 2.

39. The compound of claim 31, wherein R¹ is halogen, and ‘m’ is 1 or 2.

40. The compound of claim 39, wherein halogen is fluoro.

41. The compound of claim 31, wherein R^{a} is alkyl.

42. The compound of claim 41, wherein alkyl is neo-pentyl or iso-butyl.

43. The compound of claim 31, wherein R^{a} is cycloalkyl.

44. The compound of claim 43, wherein cycloalkyl is cyclopentyl.

45. The compound of claim 2, wherein R^{b} is methyl.

46. The compound of claim 31, wherein R^{c} is cyano.

47. The compound of claim 30, selected from:
4-{2-[(E)-2-(2,2-Dimethylpropoxy)-3-methoxyphenyl]-1-ethenyl]-4-oxo-4H-3-chromenyl}benzonitrile,
4-{7-Fluoro-2-[(E)-2-(3-methoxy-2-neopentyloxyphenyl)-1-ethenyl]-4-oxo-4H-3-chromenyl}benzonitrile,
4-{6-Fluoro-2-[(E)-2-(2-isobutoxy-3-methoxyphenyl)-1-ethenyl]-4-oxo-4H-3-chromenyl}benzonitrile
4-{6-Fluoro-2-[(E)-2-(2,2-dimethylpropoxy)-3-methoxyphenyl]-1-ethenyl]-4-oxo-4H-3-chromenyl}benzonitrile,
and
4-{2-[(E)-2-(2-Cyclopentyloxy-3-methoxyphenyl)-1-ethenyl]-6,7-difluoro-4-oxo-4H-3-chromenyl}benzonitrile
or pharmaceutically acceptable salt thereof.

48. A pharmaceutical composition comprising a compound according to claim 30, either as a free base or pharmaceutically acceptable salt form and a pharmaceutically acceptable excipient.

49. The pharmaceutical composition according to claim 48, wherein the pharmaceutically acceptable excipient is a carrier or diluent.

50. A method for preventing or treating a vanilloid receptor mediated disease, disorder or syndrome in a subject in need thereof comprising administering to the subject a therapeutically effective amount of a compound according to claim 30.

51. The method according to claim 50, wherein the vanilloid receptor in a subject is TRPV3.

52. The method according to claim 51, wherein the symptoms of a disease, disorder, syndrome or condition associated with TRPV3 function is selected from the group consisting of pain, acute pain, chronic pain, nociceptive pain, neuropathic pain, post-operative pain, dental pain, cancer pain, cardiac pain arising from an ischemic myocardium, pain due to migraine, arthralgia, neuropathies, neuralgia, trigeminal neuralgia nerve injury, diabetic neuropathy, neurodegeneration, retinopathy, neurotic skin disorder, stroke, urinary bladder hypersensitiveness, urinary incontinence, vulvodynia, gastrointestinal disorders such as irritable bowel syndrome, gastro-esophageal reflux disease, enteritis, ileitis, stomach-duodenal ulcer, inflammatory bowel disease, Crohn's disease, celiac disease, an inflammatory disease such as pancreatitis, a respiratory disorder such as allergic and non-allergic rhinitis, asthma or chronic obstructive pulmonary disease, irritation of skin, eye or mucous membrane, dermatitis, pruritic conditions such as uremic pruritus, fervescence, muscle spasms, emesis, dyskinesias, depression, Huntington's disease, memory deficits, restricted brain function, amyotrophic lateral sclerosis (ALS), dementia, arthritis, osteoarthritis, diabetes, obesity, urticaria, actinic keratosis, keratocanthoma, alopecia, Meniere's disease, tinnitus, hyperacusis, anxiety disorders and benign prostate hyperplasia.

53. A method of treating pain in a subject in need thereof comprising administering to the subject a therapeutically effective amount of a compound of claim 30.

54. The method of claim 53, wherein the pain is acute pain, chronic pain, post-operative pain or neuropathic pain.

55. A method of treating inflammation in a subject in need thereof comprising administering to the subject a therapeutically effective amount of a compound of claim 30.
